(19) United States
(12) Patent Application Publication
(10) Pub. No.: US 2026/0256501 A1
(43) Pub. Date: Sep. 3, 2026

(54) Anti-Rotation Lag Screws

(71) Applicant: Stryker European Operations Limited, Carrigtwohill (IE)

(72) Inventors: Helge Giersch, Laboe (DE); Alessia Colado Gimeno, Kiel (DE); Bernd Simon, Kiel (DE); Matthias Bartsch, Schoenkirchen (DE); Tobias Böhm, Schwentinental (DE); Manfred Wieland, Kiel (DE)

(21) Appl. No.: 19/652,685

(22) Filed: Apr. 20, 2026

Related U.S. Application Data
(63) Continuation of application No. PCT/IB2025/000499, filed on Oct. 2, 2025.
(60) Provisional application No. 63/702,435, filed on Oct. 2, 2024.

(51) Int. Cl. A61B 17/72; A61B 17/68

(52) U.S. Cl. CPC A61B 17/7241; A61B 17/683; A61B 2017/681

(57) ABSTRACT

A fracture fixation system includes an intramedullary nail having a lag screw bore extending along a lag screw bore axis and a cannulated channel extending along a cannulated channel axis and in communication with the lag screw bore, the lag screw bore axis and the cannulated channel axis extending transverse to each other, a threaded lag screw configured for insertion within a first portion of the lag screw bore, a bolt configured for insertion within a second portion of the lag screw bore, a compression screw extending within the bolt, and a set screw configured for insertion into the cannulated channel and into engagement with the compression screw and/or the bolt.

### CROSS-REFERENCE TO RELATED APPLICATIONS

The present application is a continuation of International Application No. PCT/IB2025/000499, filed Oct. 2, 2025, published in English, which claims the benefit of the filing date of U.S. Provisional Application No. 63/702,435, filed Oct. 2, 2024, the disclosures of which are hereby incorporated herein by reference.

### BACKGROUND

The present disclosure relates to bone fixation systems used to join and promote healing of fractured bone, and more particularly, but not limited to, devices used to fixate femoral fractures.

Treatment of fractures in long bones utilizing internal fixation remains challenging, especially for dislocated unstable fractures. There are a variety of devices designed to treat fractures of the femur, humerus, tibia, and other long bones. For example, fractures of the femoral neck, head, and intertrochanteric region have been treated with bone plates, external fixation devices and internal fixation systems, including intramedullary nails affixed to lag screws. While internal fixation systems provide some benefits over other means of treatment, these systems struggle with providing rotational stability of a bone head (e.g., femoral head), particularly during surgery, and maintaining the necessary compression needed to promote healing of bone fractures. Moreover, typically internal fixation systems used to treat femoral fractures generally only afford static compression without allowing for dynamic movement during the healing process.

Accordingly, there remains a need for further developments of internal fracture fixation systems to effectively and efficiently provide rotational stability and dynamic and static compression.

### SUMMARY

In one aspect, the present disclosure relates to an implant assembly that includes a bone nail and a lag screw assembly having a lag screw, a bolt, a set screw and a compression screw. The lag screw is adapted to thread into a portion of the bolt such that the bolt and the lag screw are coupled together. The bolt has a hollow portion capable of receiving a compression screw through a proximal end of the bolt and a set screw via a slot opening extending into the hollow portion. The set screw extends into the hollow portion of the bolt to prevent the compression screw from advancing past a certain point in the medial direction when implanted, while still allowing the compression screw to move laterally. The bone nail may have a straight cannulated shaft, or a bent shaft having one bend or multiple bends. In some cases, the nail may be a cephalomedullary nail. Also, the bone nail may include a singular bore or multiple bores for the lag screw, bolt and, in some case, a calcar pin. The bore or bores and internal channel(s) defined by the nail allow for the lag screw assembly to be fixed to the nail.

In some instances, the lag screw assembly may include a set screw and a locking element that extends from the set screw. The locking element is rotatably coupled to the set screw such that the set screw may be rotated independent of the set screw. When disposed within the nail, the locking element may be arranged such that the locking element does not rotate with the set screw. The locking element generally has an elongated body that extends from the set screw. In some instances, the elongated body has a tapered tip and a cannulated passage extending therethrough. The lock element may also define an elliptical opening that extends through the width of the elongated slot and is a larger than the bores defined by the nail. This allows the locking element to receive surgical tools to be inserted therethrough.

A first aspect of the present disclosure is a fracture fixation system, including an intramedullary nail having a lag screw bore extending along a lag screw bore axis and a cannulated channel extending along a cannulated channel axis and in communication with the lag screw bore, the lag screw bore axis and the cannulated channel axis extending transverse to each other, a threaded lag screw configured for insertion within a first portion of the lag screw bore, a bolt configured for insertion within a second portion of the lag screw bore, a compression screw extending within the bolt, and a set screw configured for insertion into the cannulated channel and into engagement with the compression screw and/or the bolt.

In accordance with other embodiments of the first aspect, the set screw may be disposed closer to the bolt than to the lag screw. The bolt may include an aperture extending along a longitudinal axis of the bolt. The aperture may be at least partially threaded and configured to receive the compression screw through an end of the bolt. The set screw may be monolithic.

The set screw may include a set screw body and a locking element. The set screw may be rotatably attached to the locking element. At least a portion of the locking element may be disposed within a cavity of the set screw. At least a portion of the set screw may be disposed within a cavity of the locking element. The set screw body and the locking element may be cannulated. The locking element may define an elongated slot that is obliquely oriented with respect to a longitudinal axis of the locking element.

The intramedullary nail may further define a calcar pin bore disposed between the lag screw bore and a proximal end of the intramedullary nail. A width of the elongated slot of the locking element may be larger than a width of the calcar pin bore. The calcar pin bore may be aligned with a first portion of the elongated slot of the locking element when the locking element is in a first position in the cannulated channel and with a second portion of the elongated slot of the locking element when the locking element is in a second position in the cannulated channel. The cannulated channel may have a threaded region and an unthreaded region, and the calcar pin bore may extend through the unthreaded region of the cannulated channel between the threaded region and the lag screw bore. The cannulated channel may have a first threaded region, an unthreaded region, and a second threaded region, and the calcar pin bore may extend through the unthreaded region of the cannulated channel between the first and second threaded regions.

The locking element may be configured to engage the compression screw. A distal end of the locking element may include threads configured to engage threads of the compression screw. The threads on the distal end of the locking element may be located only on a leading side of the distal end of the locking element. A trailing side of the distal end of the locking element may include a fixation surface including a plurality of ridges, the fixation surface being configured to engage an exterior surface of the bolt. The fixation surface may be concave. The threads on the leading side of the distal end of the locking element may occupy a portion of the distal end that is distinct from a portion of the distal end defining the fixation surface. When the locking element is engaged with the compression screw, the threads on the leading side of the distal end of the locking element may contact the compression screw and the fixation surface on the trailing side of the distal end of the locking element may engage an exterior surface of the bolt and cannot contact the compression screw.

The cannulated channel may extend through an entire length of the intramedullary nail. The intramedullary nail may define a bent portion disposed between proximal and distal portions. The bolt may include a thread path into which the lag screw is threaded.

A second aspect of the present disclosure is a fracture fixation system, including a nail having a first bore extending along a first bore axis and a cannulated channel extending along a cannulated channel axis and in communication with the first bore, the first bore and the cannulated channel axis extending traverse to each other, a set screw configured for insertion into the cannulated channel, a compression screw, and a first shaft and a second shaft for insertion through the first bore, the first shaft configured to be threaded to the second shaft and to receive the compression screw and a portion of the set screw.

In accordance with other embodiments of the second aspect, the compression screw may be configured to press against the set screw. The compression element may be configured to threadably engage the set screw. The first shaft may define a cylindrical cavity extending along the first bore axis, the cylindrical cavity defining a sidewall of the first shaft, the cylindrical cavity configured to receive the compression element and a counter screw rotatably attached together, and the cylindrical cavity may include a threaded region configured to threadably engage the counter screw of the compression element. The first shaft may define a cylindrical cavity extending parallel to the first bore axis, the cylindrical cavity defining a sidewall of the first shaft, the cylindrical cavity being internally threaded to receive the compression element. The first shaft may define a side slot that extends through a portion of the sidewall of the first shaft and into the cylindrical cavity. The first shaft may be configured to be placed within the first bore such that the cylindrical cavity is in communication with the cannulated channel via the first side slot. The first side slot may be dimensioned to receive a portion of the set screw. The first shaft may include a second side slot extending through the side wall of the first shaft opposite from the first side slot such that the second side slot is configured to be in communication with the cannulated channel of the nail via the first side slot.

As described above, the set screw may be monolithic or comprised of separate and distinct components. The set screw may be cannulated and may includes a locking element extending from an end of the set screw. The set screw and the locking element may be rotatably attached to each other. The side slot may be configured to receive an end of the locking element of the set screw, and the end of the locking element may be configured for insertion through the side slot of the first shaft. The locking element may include a threaded region at an end. The threaded region may be configured to threadably engage threads on the compression element. The locking element may be configured to slide along the cannulated channel axis with respect to the set screw.

A third bore may be configured to align with a portion of a through-slot defined by a locking element extending from the set screw such that both the third bore and the through-slot are configured to receive a calcar pin while the set screw is disposed in the cannulated channel. The compression element may not threadably engage the first shaft. The compression element may be configured to threadably engage the locking element and no other component.

The fracture fixation system may further include a spring disposed within the cylindrical cavity in contact with the compression element, and the compression element may be configured to rotate with respect to the counter screw and the spring. The compression element may include an opening configured to receive a portion of the spring, and the counter screw may include a central through hole configured to receive an end of an instrument adapted to engage and rotate the compression element. The third bore may be configured to align with a portion of a through-slot defined by a locking element extending from the set screw such that both the third bore and the through-slot are configured to receive a calcar pin while the set screw is disposed in the cannulated channel.

A third aspect of the present disclosure is a method of implanting a fracture fixation system, including inserting a nail into an intramedullary canal of a femur, inserting a first calcar pin and a second calcar pin through a second bore and a third bore of the nail, respectively, inserting a bolt through a first bore in the nail, removing the first calcar pin, inserting a lag screw through the second bore in the nail and into engagement with a femoral head, removing the second calcar pin, tightening a set screw in the nail and into engagement with the bolt, and advancing a compression screw through a cylindrical cavity in the bolt such that the compression screw presses against the set screw.

In accordance with other embodiments of the third aspect, tightening the set screw may include advancing the set screw into the cylindrical cavity defined by the bolt. Inserting the lag screw may include threading the lag screw into the bolt. The method may further include placing the set screw in a cannulated channel of the nail before the insertion of the second calcar pin. The set screw may include a locking element extending from an end of the set screw such that the placement of the set screw in the cannulated channel includes aligning a through-slot of the locking element with the third bore. The inserting of the second calcar pin may include inserting the second calcar pin through the through-slot of the locking element. The removal of the second calcar pin may be performed after the tightening of the set screw and the advancing of the compression screw. The tightening of the set screw may include shifting the locking element distally with respect to the second calcar pin. The removal of the second calcar pin may be performed after the tightening of the set screw and the advancing of the compression screw. The tightening of the set screw may further include advancing the set screw through the cylindrical cavity of the bolt such that the set screw engages the lag screw.

A fourth aspect of the present disclosure is an intramedullary nail including a lag screw bore extending transverse to a longitudinal axis of the nail, and a bolt bore extending transverse to the longitudinal axis of the nail, wherein the lag screw and bolt bores are located adjacent to and in communication with each other.

In accordance with other embodiments of the fourth aspect, the lag screw bore and the bolt bore may overlap to define a figure-8 shape. The nail may further include a chamfer extending from a figure-8 shaped hole in the nail to an outer surface of the nail, the figure-8 shaped hole leading to the lag screw bore the bolt bore. The nail may further include a pin bore extending transverse to the longitudinal axis of the nail and a cannulated channel extending along the longitudinal axis of the nail. The cannulated channel may include a first threaded portion and a second threaded portion separated by the pin bore. The cannulated channel may include a threaded portion adjacent to the pin bore. The nail may further include a set screw pre-loaded within the threaded portion of the cannulated channel. The nail may further include a set screw pre-loaded within the second threaded portion of the cannulated channel. A fracture fixation system may include the intramedullary nail and a threaded lag screw for insertion within the lag screw bore, and a bolt for insertion within the bolt bore, such that at least a portion of the threaded is received in a portion of the bolt.

A fifth aspect of the present disclosure is an intramedullary nail including a nail body defining a transverse bore therethrough that extends along a transverse bore axis, wherein at least a portion of an inner surface of the bore has a figure-8 shape in a plane perpendicular to the transverse bore axis.

In accordance with other embodiments of the fifth aspect, the transverse bore may be comprised of overlapped cylindrical portions that define the figure-8 shape. The overlapped cylindrical portions may include a larger cylindrical portion defined by a larger radius and a smaller cylindrical portion defined by a smaller radius. The transverse bore axis may angled with respect to a central longitudinal axis of the nail body. The transverse bore axis may be angled to substantially align with the trajectory of a central axis of a femoral neck when the intramedullary nail is implanted in a femur.

The nail body may further include a pin bore therethrough that extends along a pin bore axis. The pin bore may be proximal of the transverse bore. The pin bore axis may be parallel to the transverse bore axis. The pin bore axis may not be parallel to the transverse bore axis such that the pin bore axis and the transverse bore axis form an acute angle and converge toward a head of a femur when the intramedullary nail is implanted in a femur. The pin bore axis may diverge from the transverse bore axis in a direction toward a head of a femur when the intramedullary nail is implanted in a femur.

A sixth aspect of the present disclosure is a method of implanting a fracture fixation system, including inserting a nail into an intramedullary canal of a femur, inserting a first calcar pin and a second calcar pin through a second bore and a third bore of the nail, respectively, inserting a bolt through a first bore in the nail, removing the first calcar pin, inserting a lag screw through the second bore in the nail and into engagement with a femoral head, removing the second calcar pin, advancing a set screw into the nail and into engagement with threads of a compression screw disposed within a cylindrical cavity of the bolt, and rotating the compression screw within the cylindrical cavity such that the bolt moves laterally with respect to the set screw.

In accordance with other embodiments of the sixth aspect, advancing the set screw may include advancing the set screw into the cylindrical cavity defined by the bolt. Inserting the lag screw may include threading the lag screw into the bolt. The method may further include placing a set screw in a cannulated channel of the nail before the insertion of the second calcar pin. The set screw may include a locking element extending from an end of a swivel screw such that the placement of the set screw in the cannulated channel includes aligning a through-slot of the locking element with the third bore.

The inserting of the second calcar pin may include inserting the second calcar pin through the through-slot of the locking element. The removal of the second calcar pin may be performed after the advancing of the set screw and the rotating of the compression screw. The advancing of the set screw may include shifting the locking element distally with respect to the second calcar pin. The removal of the second calcar pin may be performed after the advancing of the set screw and the rotating of the compression screw. The method may further include partially retracting the set screw while still maintaining the engagement between the set screw and the compression screw. The method may further include tightening the set screw down against the compression screw to establish a static locking configuration after the rotating of the compression screw.

### DESCRIPTION OF THE DRAWINGS

A more complete appreciation of the subject matter of the present disclosure and the various advantages thereof may be realized by reference to the following detailed description which refers to the accompanying drawings, in which:

FIG. 1 is a cross-sectional view of a fixation assembly in accordance with an aspect of the present disclosure;

FIG. 2a is a cross-sectional view of an intermedullary nail of the fracture fixation assembly of FIG. 1;

FIG. 2b is a side view of the intramedullary nail of FIG. 2;

FIG. 3 is a perspective view of a bolt and a lag screw of the fixation assembly of FIG. 1;

FIG. 4 is a side view of the lag screw of FIG. 3;

FIG. 5 is another side view of the lag screw of FIG. 3;

FIG. 6 is a perspective view of a compression screw of the fixation assembly of FIG. 1;

FIG. 7 is a perspective view of a set screw of the fixation assembly of FIG. 1;

FIG. 8a is a cross-sectional view of a fixation assembly in accordance with another aspect of the present disclosure;

FIG. 8b is a close-up view of the fixation assembly of FIG. 8a;

FIG. 9a is a perspective view of a set screw of the fixation assembly of FIG. 8a,

FIG. 9b is a side view of the set screw of FIG. 9a;

FIG. 10 is a perspective view of swivel screw of the set screw of FIG. 9a;

FIG. 11 is a perspective view of a locking element of the set screw of FIG. 9a;

FIG. 12 is a perspective view of a lag screw, a bolt, and the set screw of the fixation assembly of FIG. 8a;

FIG. 13 is a cross-sectional view of a fixation assembly in accordance with an aspect of the present disclosure;

FIG. 14 is a close-up view of the fixation assembly of FIG. 13;

FIG. 15 is a close-up view of the fixation assembly of FIG. 13 in a different configuration;

FIG. 16 is a close-up view of the fixation assembly of FIG. 14;

FIG. 16A is a perspective view of the fixation assembly of FIG. 14;

FIG. 16B is a close-up cross-sectional view of a portion of the fixation assembly of FIG. 16;

FIG. 17 is a perspective view of a locking element of a set screw of the fixation assembly of FIG. 13;

FIG. 18 is a side view of the locking element of FIG. 18;

FIG. 19 is a cross-sectional view of a compression screw and an end screw of the fixation assembly of FIG. 13;

FIG. 20 is a perspective view of the compression screw and the end screw of FIG. 19;

FIG. 21 is a cross-sectional view of the end screw of FIG. 19;

FIG. 22 is a cross-sectional view of a fixation assembly in accordance with an aspect of the present disclosure;

FIG. 23 is a side view of a set screw of the fixation assembly of FIG. 13;

FIG. 24 is a cross-sectional view of a fixation assembly in accordance with another aspect of the present disclosure;

FIG. 25a is a cross-sectional view of a screw element and a locking element of the fixation assembly of FIG. 15;

FIG. 25b is a cross-sectional view of the screw element and the locking element of the fixation assembly of FIG. 15;

FIG. 26 is a side view of the locking element of the set screw of the fixation assembly of FIG. 15;

FIG. 27 is a cross-sectional view of the set screw of the fixation assembly of FIG. 15;

FIG. 28 is a cross-sectional view of a fixation assembly in accordance with an aspect of the present disclosure;

FIG. 29 is a cross-sectional view of a set screw of the fixation assembly of FIG. 28;

FIG. 30 is a cross-sectional view of a compression screw of the fixation assembly of FIG. 28;

FIG. 31 is a cross-sectional view of a bolt of the fixation assembly of FIG. 28;

FIG. 32 is a cross-sectional view of a fixation assembly in accordance with an aspect of the present disclosure;

FIG. 33 is a perspective of a fixation assembly in accordance with an aspect of the present disclosure;

FIG. 34 is a cross-sectional view of the fixation assembly of FIG. 33;

FIG. 35 is a close-up view of a locking element and a bolt of the fixation assembly of FIG. 33;

FIG. 36 is a bottom view of the locking element of FIG. 35;

FIG. 37 is a perspective view of the locking element of FIG. 33;

FIG. 38 is a close-up cross-sectional view of a portion of the fixation assembly of FIG. 33;

FIGS. 39-46 illustrate steps for implanting and/or assembling a fixation assembly in accordance with an aspect of the present disclosure; and

FIGS. 47-53 illustrate steps for implanting and/or assembling a fixation assembly in accordance with another aspect of the present disclosure.

### DETAILED DESCRIPTION

The present disclosure describes fixation assemblies for fixation of bone fractures. Particularly, the fixation assemblies include at least a nail and a lag screw assembly configured to engage with the nail. The lag screw assembly includes a lag screw having a shank and a bolt adapted to engage with a thread defined by the shank. The nail is configured to be inserted into a bone, such as the intramedullary canal of a femur, and the components of the lag screw assembly are further configured to be inserted into the bone and passed through a bore or bores defined by the nail to thereby couple the lag screw assembly and the nail. It should be understood that the fracture fixation system described herein may be applied to long bones in general, and while specifically designed for use in fixing a femoral head fracture in the present description, it may also be designed for use in a humerus, tibia and other long bones.

As used herein, the term “proximal,” when used in connection with a device or components of a device, refers to the end of the device closer to the user of the device (e.g., surgeon or operator) when the device is being used as intended. On the other hand, the term “distal,” when used in connection with a device or components of a device, refers to the end of the device farther away from the user (e.g., surgeon or operator) when the device is being used as intended. As used herein, the term “superior” refers to an upward direction on the page or relative to an anatomy of a person standing upright. On the other hand, the term “inferior” refers to a downward direction on the page or relative to an anatomy of a person standing upright. It should be understood that these terms are not limiting, but merely used for ease of description, and that varied orientations may cause directions to differ. As used herein, the terms “substantially,” “generally,” “approximately” and “about” are intended to mean that deviations from absolute are included within the scope of the term so modified.

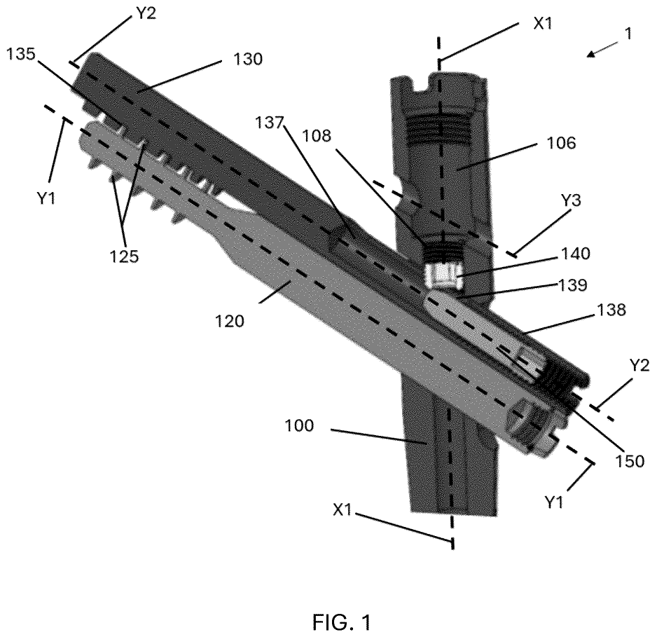

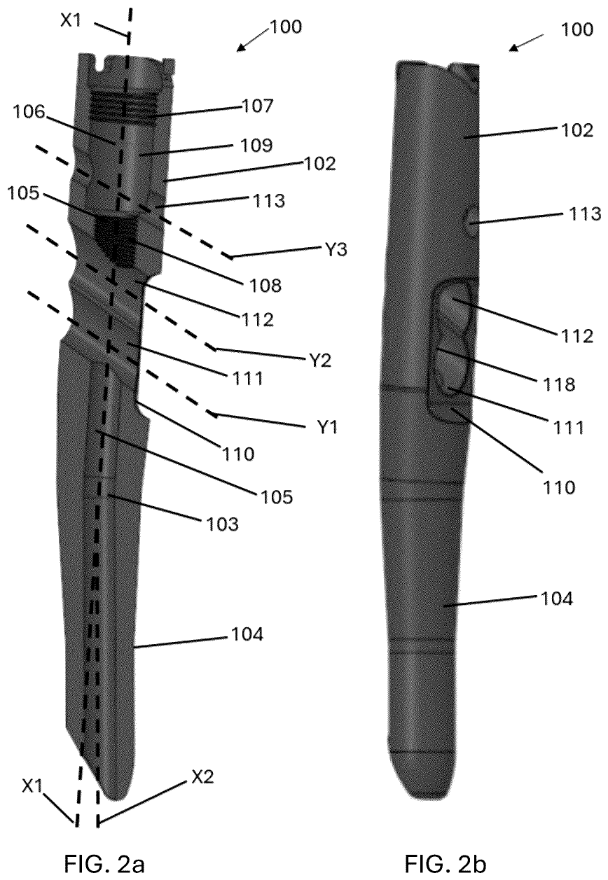

Now referring to FIGS. 1-7, one aspect of the disclosure relates to a first fixation assembly 1 including an intramedullary nail 100, a lag screw 120, a bolt 130, a set screw 140, and a compression screw 150. Intramedullary nail 100 includes a bend 103 disposed between a proximal portion 102 and a distal portion 104 of the nail. The proximal portion extends along a proximal axis X1, and the distal portion extends along a distal axis X2. Proximal axis X1 and distal axis X2 intersect at bend 103 which defines the angle between the proximal and distal axes X1, X2. Distal portion 104 of nail 100 is tapered from bend 103 to a terminal end of the nail. A cannulated channel 105 extends through the length of the nail 100 such that the channel extends along the proximal axis X1 in the proximal portion 102 and the distal axis X2 in the distal portion 104. Cannulated channel 105 defines a set screw portion 106 having a larger diameter than the remainder of the cannulated channel. Set screw portion 106 of cannulated channel 105 has a first thread portion 107 and a second threaded portion 108 that are separated by unthreaded portion 109. Proximal portion 102 defines a side groove 110 extending partially into the outer surface of nail 100 and having a U-shaped cross-sectional profile, as shown in FIG. 2b.

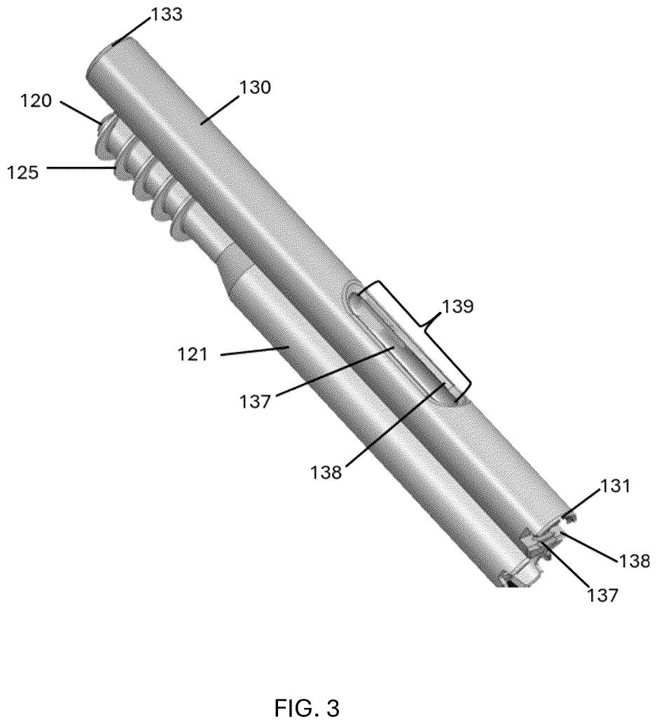

Intramedullary nail 100 further defines a lag screw bore 111, a bolt bore 112, and a pin bore 113 that extend generally transversely therethrough along a lag screw bore axis Y1, bolt bore axis Y2 and a pin bore axis Y3, respectively, such that the bores are aimed along the neck and into the head of a femur when the nail is implanted within the intramedullary canal of the femur. Lag screw bore 111 and bolt bore 112 extend through side groove 110. Pin bore 113 extends through unthreaded portion 109 of cannulated channel 105. Lag screw bore 111, bolt bore 112 and pin bore 113 are each generally rounded defining a cylindrical shape. Lag screw bore 111 and the bolt bore 112 overlap each other to form a double-bore slot 114 in the nail. In other words, lag screw bore 111 and bolt bore 112 generally define a figure-8 shape 118 in a plane perpendicular to lag screw bore axis Y1 and/or bolt bore axis Y2 that extends through an entire thickness of the nail 100 as shown in FIG. 3. Lag screw bore axis Y1, bolt bore axis Y2 and pin bore axis Y3 intersect proximal axis X1 at an oblique angle. It is contemplated that the lag screw bore 111, bolt bore 112, and/or pin bore 113 may be defined such that their respective bore axes (i.e., Y1, Y2, Y3) are orthogonal to proximal axis X1, or such that any one of the bore axes Y1, Y2, Y3 may form an acute or obtuse angle with the axis X1. While the nail 100 is described and illustrated as having certain features, it is further contemplated that intramedullary nail may have different configurations such as straight or may contain additional bends along its length.

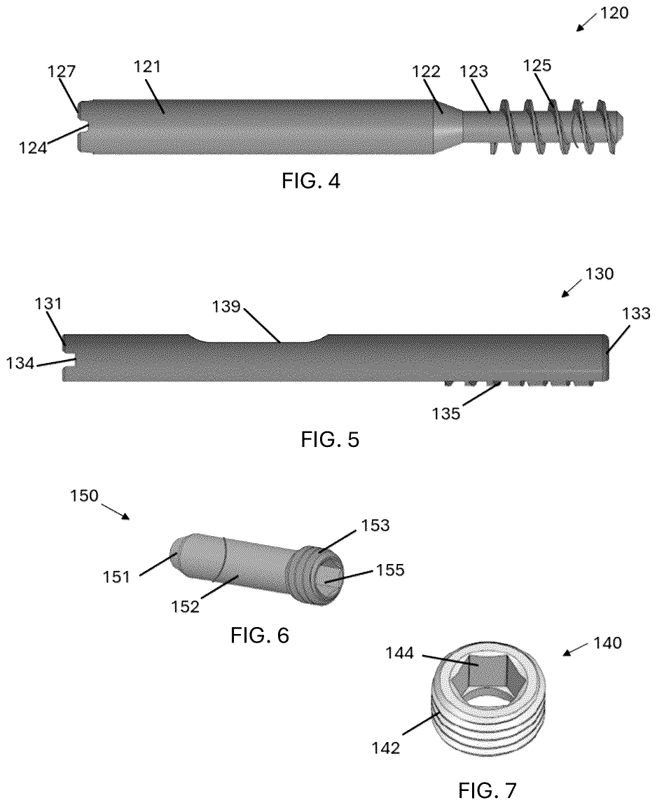

Lag screw 120 defines shaft portion 121, a tapered portion 122, and a threaded portion 123, as shown in FIG. 4. Shaft portion 121 has a generally constant outer diameter along its length and includes a proximal end 127 defining a cavity and notches 124 configured to be engaged by instrumentation designed for inserting and rotating lag screw 120 for implantation. The outer diameter of shaft portion 121 is less than or equal to the inner diameter of lag screw bore 111 such that lag screw 120 can be inserted therethrough. Threaded portion 123 defines thread 125 extending from the exterior surface of the threaded portion of lag screw 120. Thread 125 defines an outer diameter that is less than or equal to the outer diameter of shaft portion 121.

Bolt 130 extends between a proximal end 131 and a distal end 133. Proximal end 131 of bolt 130 defines a cylindrical cavity 137 and notches 134 configured to be engaged by instrumentation designed for inserting and positioning the bolt through bolt bore 112 of nail 100. Cylindrical cavity 137 defines a side wall 138 of bolt 130 which has a side slot 139 extending therethrough, as shown in FIG. 1. Side slot 139 is dimensioned to receive a portion of set screw 140 extending through cannulated channel 105 of nail 100 (discussed in more detail below) when implanted. Cylindrical cavity 137 is partially threaded such that compression screw 150 may be threaded therein (also discussed in more detail below). Bolt 130 further defines a thread path 135 that corresponds to thread 125 of lag screw 120. Thread path 135 extends from the outer surface of bolt 130 near distal end 133 and defines an outer diameter, along with the remainder of the bolt, that is less than the inner diameter of bolt bore 112 such that the bolt can be inserted therethrough. This permits lag screw 120 and bolt 130 to be implanted closer together in the overall construct.

FIG. 6 illustrates compression screw 150, which has a tapered tip 151, an unthreaded shaft 152 and a threaded head portion 153 defining hexagonally shaped cavity 155 configured to receive an insertion tool. FIG. 7 depicts a canulated set screw 140, which has external threads 142 and a hexagonally shaped inner surface 144. It is contemplated that the set screw may be a monolithic part or a multi-component part including a screw element/body and a locking element that are separate and discrete from one another, as described in more detail below. In either case, the set screw is designed to be threaded into and advanced through a threaded channel in an intramedullary nail.

When fully assembled, as shown in FIG. 1, the first fixation assembly 1 is arranged such that lag screw 120 and bolt 130 are disposed within lag screw bore 111 and bolt bore 112, respectively. in this state, thread 125 of lag screw 120 is threaded into thread path 135 of bolt 130. Bolt 130 is disposed within bolt bore 112 with cylindrical cavity 137 in communication with cannulated channel 105 via side slot 139. In other words, bolt 130 is positioned within bolt bore 112 with side slot 139 facing second threaded portion 108 of cannulated channel 105, as shown in FIG. 1, which allows set screw 140 to extend from the set screw portion of cannulated channel 105 into cylindrical cavity 137. Set screw 140 is threaded to second threaded portion 108 of the cannulated channel. Screw set 140 may also engage a portion of bolt 130. Compression screw 150 is disposed within cylindrical cavity 137 of bolt 130 such that the compression screw is pressed against an end or edge of set screw 140 and thereby prevented from further advancement toward a femoral head. In this manner, when first fixation assembly 1 is implanted, lag screw 120 and bolt 130 are static with respect to movement toward a femoral head but are dynamic with respect to movement away from the femoral head along lag screw bore axis Y1 and bolt bore axis Y2, respectively.

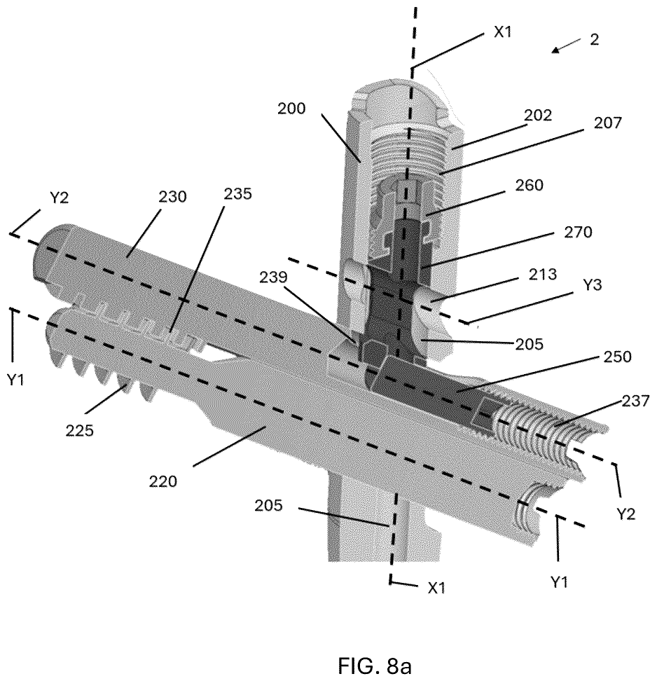

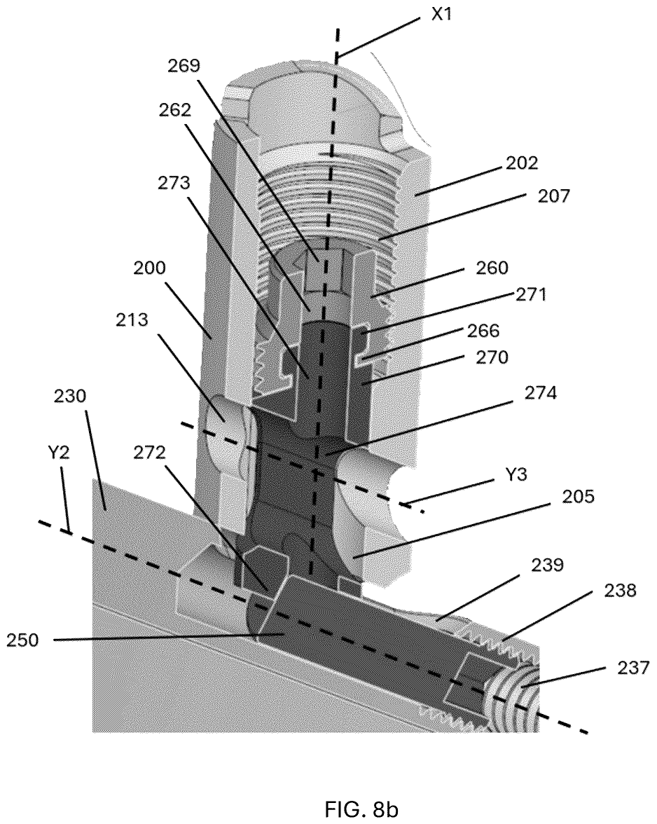

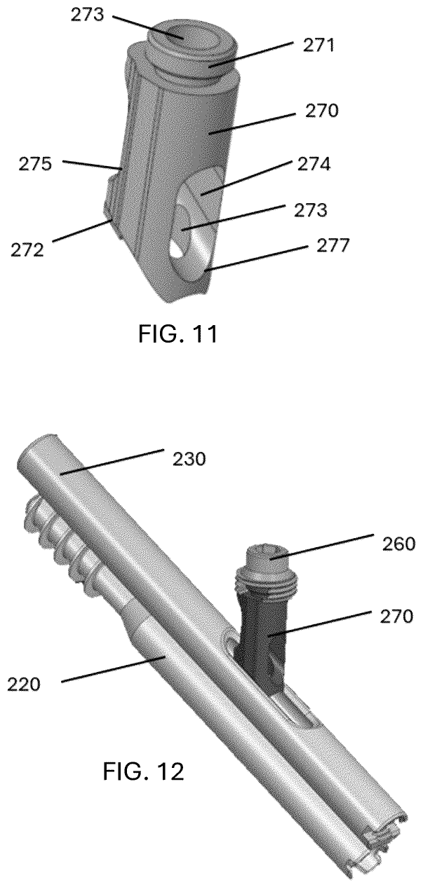

Now referring to FIGS. 8a-12, another aspect of the present disclosure relates to a second fixation assembly 2 that includes intramedullary nail 200, lag screw 220, bolt 230, compression screw 250, set screw body or swivel screw 260, and locking element 270. Unless otherwise described, it should be understood that the 200-series reference numerals correspond with the previous series references numerals to indicate components having the same or similar features as described above. Like with first fixation assembly 1, nail 200 defines lag screw bore 211, bolt bore 212 and pin bore 213. Pin bore 213 has a diameter large enough to receive a bone pin such as a calcar pin or other types of compression pins. Swivel screw 260 has cannulated passage 262 and defines a threaded portion 261 and extension portion 263. Extension portion 263 has a smaller diameter than that of threaded portion 261 and defines hexagonally shaped inner surface 269 arranged to be engaged by insertion and rotation tools. Threaded portion 261 has external threads 267 and a housing portion 265 extending internally from an end of extension portion 263 to a lip 266 within swivel screw 260. Housing portion 265 defines a diameter larger than that of cannulated passage 262 and a side opening 268 extending through external threads 267.

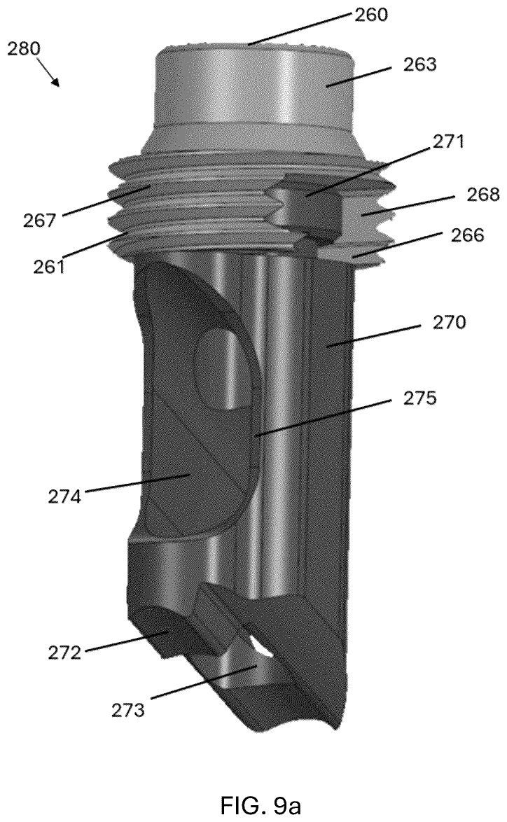

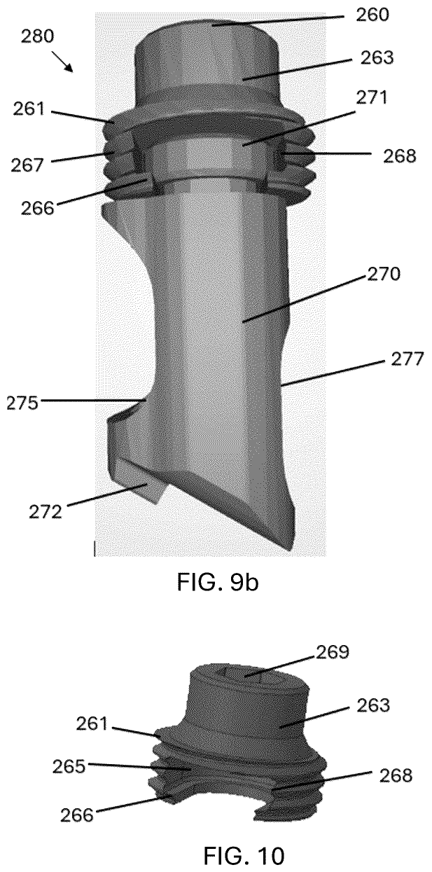

Locking element 270 includes a circular flange 271 at one end and a projection 272 at an opposite end thereof. Locking element 270 defines a cylindrical channel 273 extending through the length of the locking element along central axis that is coaxial or parallel with proximal axis X1 when disposed within cannulated channel 205. Elongated slot 274 extends through the width of the locking element 270 and thus through cylindrical channel 273 at an oblique angle relative the central axis of the cylindrical channel. Elongated slot 274 defines an opening that is wider than the diameter of pin bore 213 of nail 200, as shown in FIG. 8b. Locking element 270 further defines a first groove 275 that extends partially into one side of the locking element and a second groove 277 that extends partially into another side 278 of the locking element such that the first and the second grooves have U-shaped profile, as shown in FIG. 9b, and are opposite from each other. When locking element 270 is disposed within a nail, first groove 275 and second groove 277 may prevent rotation of the locking element therein. Circular flange 271 has a diameter that is smaller than the inner diameter of the housing portion 265 and the width of side opening 268 such that the circular flange can be inserted and rotated therein. When circular flange 271 is disposed within housing portion 265 such that swivel screw 260 and locking member 270 are rotatably connected, set screw 280 is formed. To assemble locking element 270 and swivel screw 260, flange 271 of the locking element must be inserted into side opening 268 of the swivel screw such that the flange is disposed between extension portion 263 and lip 266, as shown in FIGS. 9a and 9b.

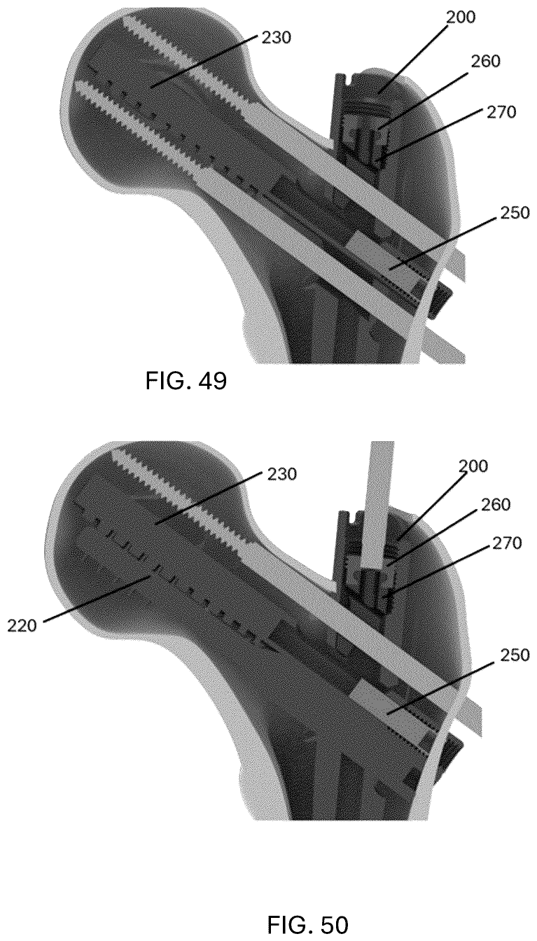

Second fixation assembly 2 is assembled in a similar manner as the first fixation assembly 1 with a few exceptions relating to swivel screw 260 and locking element 270. For example, locking element 270 of set screw 280 extends from cannulated channel 205 to cylindrical cavity 237, and swivel screw 260 is threaded into first threaded portion 207 of the cannulated channel. A portion of elongated slot 274 of locking element 270 is aligned with pin bore 213 of nail 200 such that a calcar pin could be inserted therethrough, as shown in FIG. 49, along pin bore axis Y3. Projection 272 of locking element 270 is disposed within cylindrical cavity 237 of bolt 130 such that the compression screw is pressed against the projection of the locking element and thereby prevented from further advancement toward a femoral head. Similar to the first fixation assembly 1, lag screw 220 and bolt 230 of second fixation assembly 2 are static with respect to movement toward a femoral head but dynamic with respect to movement away from the femoral head along lag screw bore axis Y1 and bolt bore axis Y2, respectively.

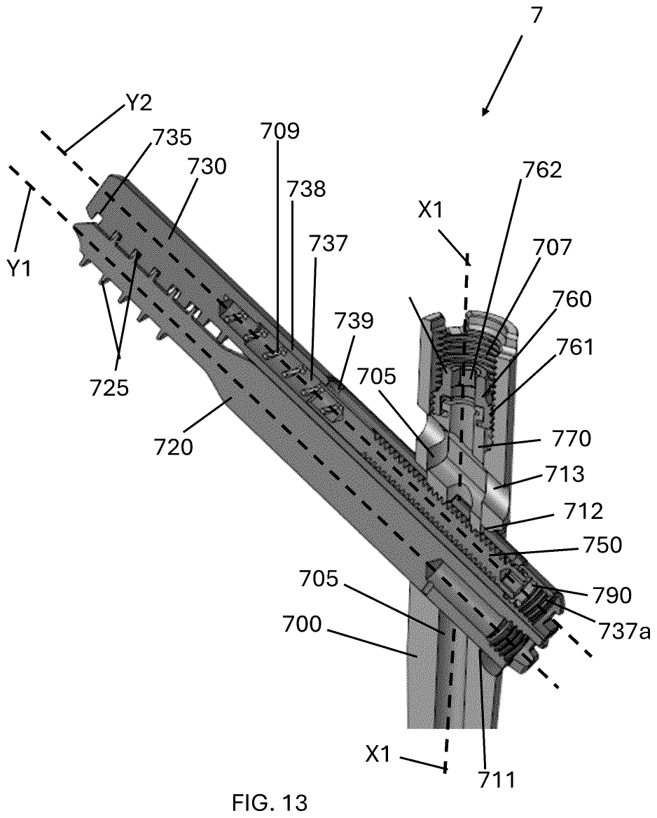

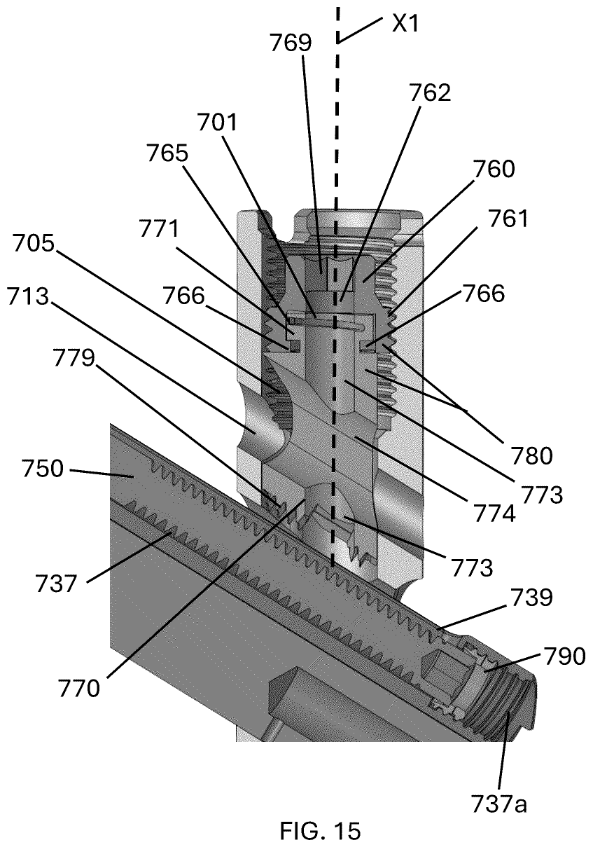

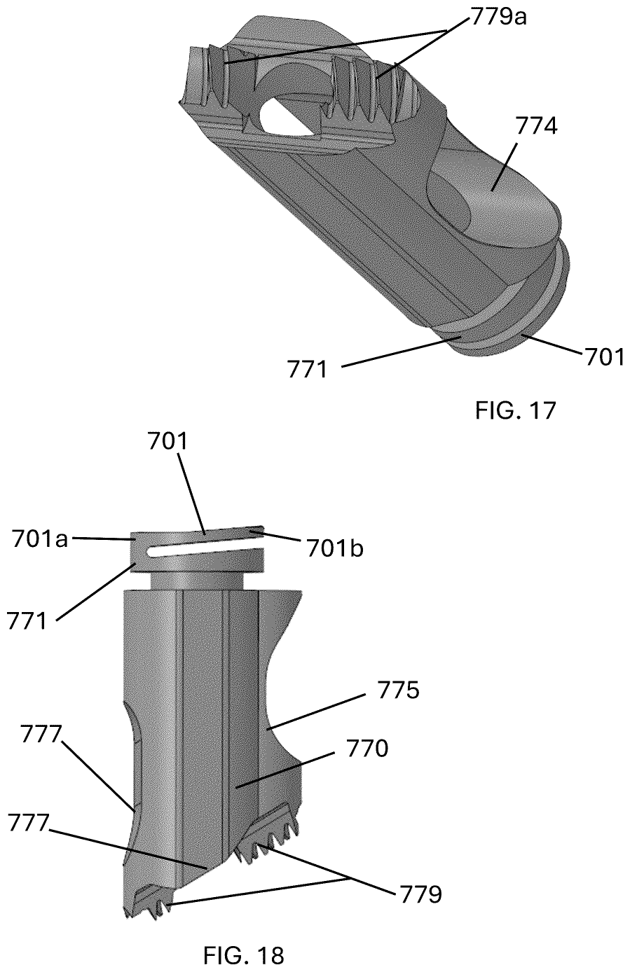

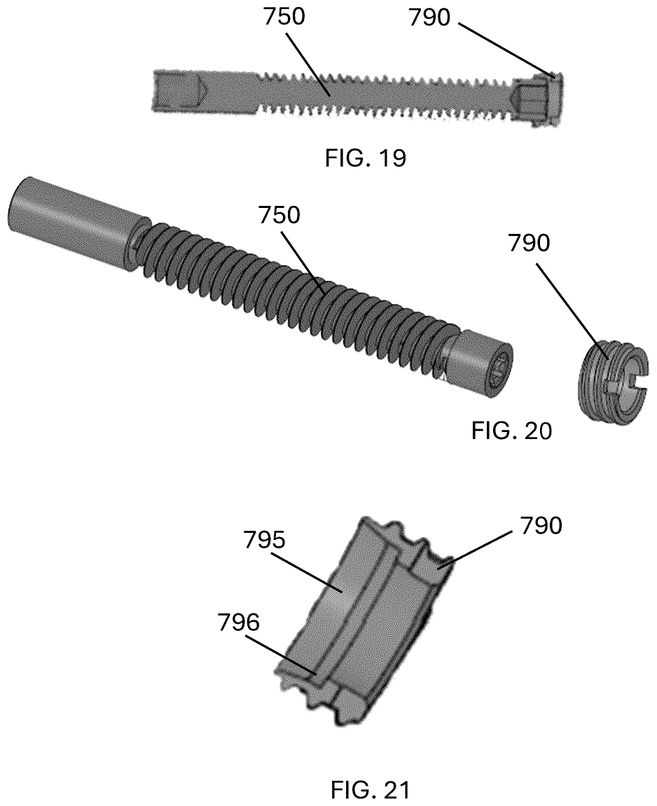

Now referring to FIGS. 13-21, another aspect of the present disclosure relates to another fixation assembly 7 that includes intramedullary nail 700, a lag screw 720, bolt 730, compression screw 750, swivel screw 760, and locking element 770 having a circular flange 771. Unless otherwise described, it should be understood that the 700-series reference numerals correspond with the previous series'references numerals to indicate components having the same or similar features as described above. Like with the first and second fixation assemblies 1 and 2, nail 700 defines lag screw bore 711, bolt bore 712 and pin bore 713. Pin bore has a diameter large enough to receive a bone pin such as a calcar pin or other types of compression pins. Swivel screw 760 and locking element 770 make up a set screw 780. Swivel screw 760 has an extension portion 763, a threaded portion 761, and a cannulated passage 762 extending through the swivel screw. Extension portion 763 of the swivel screw 760 has a smaller diameter than that of threaded portion 761 and defines hexagonally shaped inner surface 769 arranged to be engaged by insertion and rotation tools. Threaded portion 761 has external threads 767 and a housing portion 765 located between the extension portion 763 and an inner lip 766 of the swivel screw 760. Housing portion 765 defines a diameter larger than that of cannulated passage 762 to accommodate circular flange 771 of locking element 770, as shown in FIG. 15.

Bolt 730 defines a cavity 737 having a threaded opening 737a and configured to receive compression screw 750 and an end screw 790. The end screw 790 has an external thread 792 and a bottom opening 795. The external thread 792 of the end screw 790 has a larger major diameter (i.e., outer diameter) than that of the threads of the compression screw 750. The external thread 792 of the end screw 790 is designed to be threaded into the threaded opening 737a of the cavity 737 of the bolt 730. Additionally, the width or diameter of the cavity 737 of the bolt 730 is larger than the major diameter of the threads of the compression screw 750 so that the threads of the compression screw do not directly engage with the bolt 730, including the threaded opening 737a. The cavity 737 of the bolt 730 further includes a narrow end 737b located at the cavity opposite of the threaded opening 737a. In many instances, the threaded opening 737a defines the largest inner diameter of the cavity 737 of the bolt and the narrow end 737b of the cavity 737 defines the smallest inner diameter of the cavity 737. The narrow end 737b has a width or diameter that is smaller than the outer diameter of the compression screw 750 so that the compression screw cannot be inserted therein. The bolt 730 also includes a side slot 739 that is in communication with the cavity 737 of the bolt 730 and is positioned closer to the threaded opening 737a than the narrow end 737b. In this manner, when the compression screw 750 is disposed within the cavity 737 of the bolt 730, the threads of the compression screw 750 are accessible via the side slot 739 of the bolt 730.

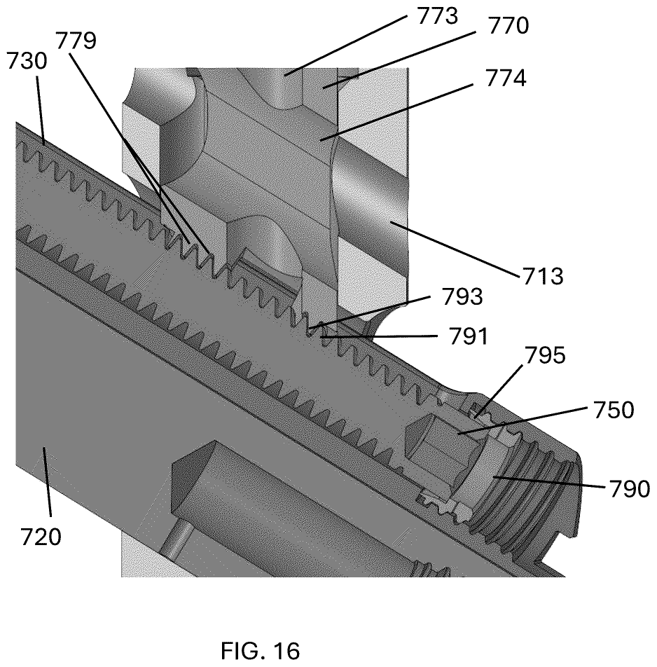

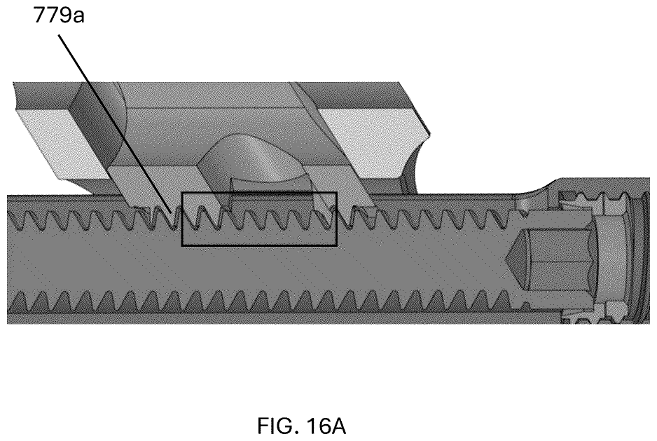

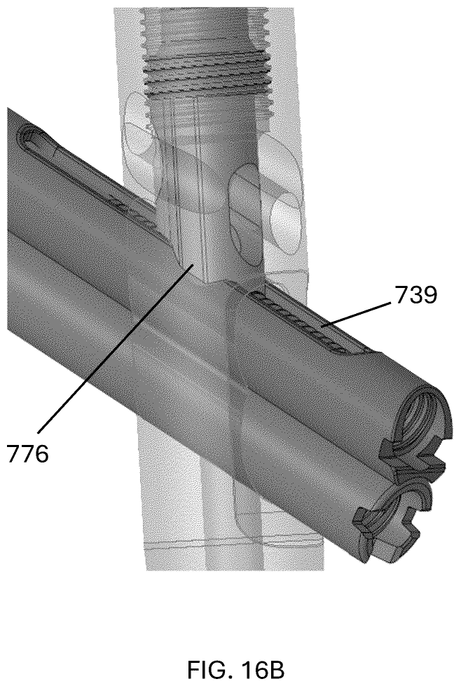

The bottom opening 795 of the end screw 790 is sized and dimensioned to receive an end of the compression screw 750, as shown in FIG. 16. The compression screw 750 is not threadedly attached to the end screw 790 and may therefore freely rotate with respect to the end screw 790 when an end of the compression screw is positioned within the bottom opening 795 of the end screw. Adjacent bottom opening 795, an internal wall of end screw 790 defines a shoulder 796 that abuts against the end of compression screw 750 configured to be disposed within bottom opening 795. In this way, threading end screw 790 into the threaded opening 737a of the cavity 737 of the bolt 730 allows shoulder 796 to act as an end stop for compression screw 750. It is also contemplated that the compression screw 750 may be rotatably attached to the end screw 790. This may be done similar to how the swivel screw and the locking element are rotatably attached together, as described herein. Additionally, the end screw 790 is cannulated such that a tool used for rotating the compression screw 750 may be inserted through the end screw to rotate the compression screw. The end screw 790 itself is inserted using a flat-end driver that interacts with two notches on the lateral end of end screw 790, as shown in FIG. 20. The internal surface of end screw 790 can be cylindrical and smooth so that it does not inadvertently interfere or interact with the driver used to manipulate compression screw 750.

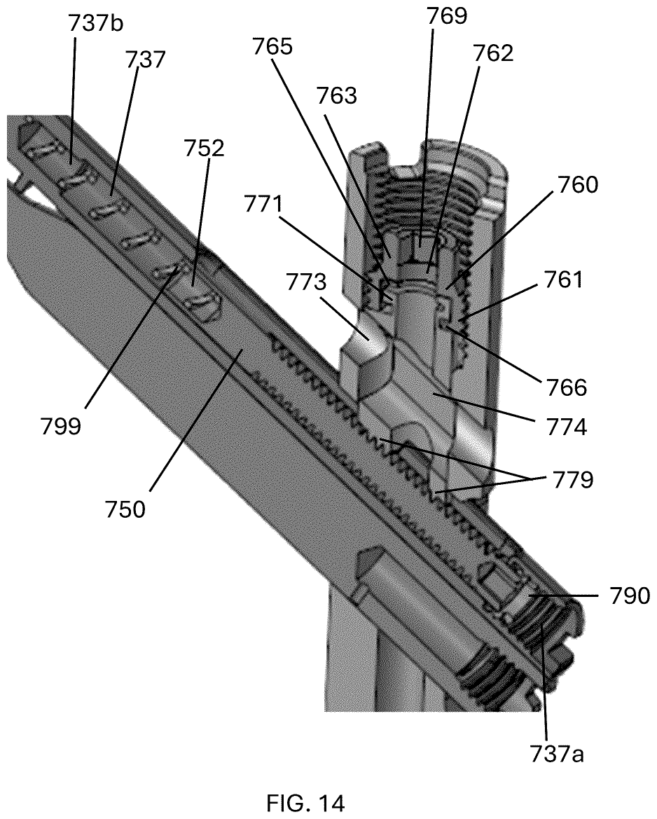

A spring 799 is placed inside the cavity 737 of the bolt 730 opposite of end screw 790 such that at an end of the spring 799 is located within the narrow end 737b of the cavity 737. The compression screw 750 defines a receiving cavity 752 that is sized and dimensioned to receive the other end of the spring 799, as shown in FIG. 13. The width or diameter of the receiving cavity 752 may be the same as or similar to the width or diameter of the narrow end 737b. The compression screw 750 is positioned between the spring 799 and the end screw 790, as shown in FIGS. 13 and 14, such that the spring is arranged and adapted to push the compression screw 750 toward and into the bottom opening 795 of the end screw 790. In this manner, the spring 799 properly biases and positions the compression screw 750 with respect to the side slot 739 and the end screw 790 without restricting or inhibiting the compression screw's ability to freely rotate within the cavity 737 of the bolt 730, as shown in FIG. 14.

Locking element 770 defines a cylindrical channel 773 extending through the length of the locking element along its central axis that is coaxial with proximal axis X1 when the locking element is disposed within the cannulated channel 705 of the nail 700. Elongated slot 774 extends through the width of the locking element 770 and traverses the cylindrical channel 773 at an oblique angle relative the central axis of the cylindrical channel. Elongated slot 774 defines an opening that is wider than the diameter of pin bore 713 of nail 700, as described above. Locking element 770 further defines a first groove 775 that extends partially into one side of the locking element and a second groove 777 that extends partially into another side of the locking element such that each of the first and the second grooves have a concave profile, as shown in FIG. 18, and are opposite from each other. These grooves help create a non-circular cross section of the external surface of locking element 770 that cooperates non-circular cross section of the internal bore of the nail in which locking element 770 is disposed, so that locking element 770 does not rotate within the nail during actuation of set screw 780.

Locking element 770 also includes a circular flange 771 at one end and a grooved or threaded end portion 779 at another end opposite of the circular flange. The threaded end portion 779 defines a thread pattern 779a configured to engage and interface with the threads of compression screw 750, as shown in FIGS. 16 and 16A. In some instances, the threaded end portion 779 may have a concave structure with a radius matching the curvature of the compression screw 750. The threaded end portion 779 is divided into two segments as the cylindrical channel 773 extends therethrough. It is contemplated, however, that in some instances the cylindrical channel 773 may not extend entirely through the locking element 770. In such instances, the thread pattern 779a of the threaded end portion 779 would be uninterrupted.

The circular flange 771 defines an outer diameter that is smaller than the inner diameter of the housing portion 765 of the swivel screw 760 and the width of the side opening thereto such that the circular flange may be inserted and rotated within the swivel screw. The circular flange 771 is received within housing portion 765 of the swivel screw 760 such that swivel screw and locking member 770 are rotatably connected to each other and to form the set screw 780. In this manner, the swivel screw 760 may be rotated independent of the locking element 770 allowing the set screw 780 to advance through the nail 700 as the swivel screw is threaded into and advanced along the first threaded portion 707 of the cannulated channel 705 of the nail 700. As indicated above, rotation of swivel screw 760 does not result in rotation of locking element 770 in the same manner due to the outer surface of locking element 770 and its interaction with the nail.

In some instances, circular flange 771 may include a cantilever arm or spring 701 extending from or constituting part of the circular flange. The cantilever spring 701 may have a first side 701a that is attached to the circular flange 771 and a second side 701b, opposite from the first side, that is unattached and extends away from the circular flange 771 such that the second side of the cantilever spring 701 is positioned further from the central flange 771 than the first side of the cantilever spring, as shown in FIG. 18. In other words, the cantilever spring 701 and the circular flange 771 define a V-shaped or U-shaped formation such that there is a gap between the second side 701b of the cantilever spring 701 and the circular flange 771 of the locking element 770. In such instances, the second side 701b of the cantilever spring 701 of the circular flange 771 must be pressed toward the circular flange 771, reducing the gap therebetween, in order to fit the circular flange 771 and the cantilever spring 701 into the housing 765 of the swivel screw 760, as shown in FIG. 15. When cantilever spring 701 is positioned within the housing 765, the second side 701b of the cantilever spring 701 expands to its unbiased or equilibrium position and thereby is pressed against an upper surface of the housing 765 of the swivel screw 760, which helps the circular flange 771 hold itself in place within swivel screw 760. This also causes the circular flange 771 to be pressed against the lip 766 of the swivel screw 760. In this manner, the cantilever spring 701 of circular flange 771 provides a tight fit and secure connection between the locking element 770 and the swivel screw 760 when assembled to form the screw 780.

Fixation assembly 7 may be assembled in a substantially similar way as the first and second fixation assemblies 1 and 2 with a few exceptions relating to swivel screw 760, locking element 770, and compression screw 750. For example, the set screw 780 may be inserted and rotated through the cannulated channel 705 of the nail 700 such that the threaded end portion 779 of the locking element 770 is advanced into the cavity 737 of the bolt 730 and the threads 779a of the threaded end portion 779 engage the threads of the compression screw 750, as shown in FIG. 14. To achieve this, the threaded end portion 779 of locking element 770 is advanced through the side slot 739 of the bolt 730 to access the cavity 737 thereof. In this engaged configuration between the locking element 770 and the compression screw 750, rotation of the compression screw 750 moves the compression screw with respect to the locking element 770. As the locking element 770 is fixed within the nail 700 in a medial-lateral direction and the compression screw 750 is retained within the bolt 730 by the end screw 790, rotating the compression screw will shift the bolt and the lag screw laterally with respect to the nail. The force of compression screw 750 against end screw 790, which is threaded into bolt 730, causes the bolt 730 and the threadedly connected lag screw 720 to also move laterally to create compression on the bone fracture. In this manner, rotation of the compression screw when it is engaged by the threaded end portion of the locking element results in bone fragments being drawn together (via compressive forces) as the bolt and lag screw shift with respect to the nail. Compression screw 750 is configured with the left-handed thread so that clockwise rotation of compression screw 750 moves it laterally with respect to locking element 770. Since end screw 790 has a right-handed thread, this clockwise rotation of compression screw 750 does not affect the anchored position of end screw 790 within bolt 730. The length of the side slot 739 in the bolt 730 stops the lateralization as kind of medial endstop, as the set screw is inserted into to the side slot 739 and hits the medial side of the side slot 739. During healing, compression screw 750 may be permitted to move away from end screw 790 slightly since the elements are not translationally locked. Thus, compression screw 750 may move medially, either solely against spring 799 or possibly to move bolt 730 and lag screw 720, which can allow micromotion and settling of the bone fracture to promote healing. In some embodiments, spring 799 is strong enough to prevent micromotion or medial movement altogether. When assembly 7 is at rest, spring 799 holds compression screw 750 firmly in place against end screw 790, particularly during and prior to implantation of bolt 730 to ensure proper positioning of compression screw 750 for engagement with threaded end portion 779 of locking element 770. The design of system 7 gives the option to over insert the bolt 730 and apply compression until the lateral sides of the bolt 730 and the lag screw 720 are flush with the lateral outer cortex of the femoral bone.

The threads on compression screw 750 allow static locking at all locations, as they are deeper to create more contact and friction with the relatively deeper threads on threaded end portion 779 of locking element 770. In addition, if static locking is necessary, the set screw can be tightened until a surface, which may have some sharp edges or ridges as described below in connection with assembly 8, hits the outer surface of the bolt 730. These surfaces 776 are at the anterior and posterior sides of locking element 770 and extend downward toward threaded end portion 779 to contact a side of the bolt 730 adjacent to side slot 739 as shown in FIG. 16B, for example. Due to the high friction in this configuration, the movement of the bolt 730 together with the lag screw 720 and head is not possible.

One additional feature of assembly 7 that can ensure proper operation during active compression is an asymmetric design of the screw threads on the compression screw 750 and the threaded end portion 779 of the locking element 770. As can be seen in FIGS. 16 and 16A, the profile of the individual threads 791 of compression screw 750 are tilted medially so that their medially-facing (leading) surfaces are closer to perpendicular to the central axis of compression screw 750 as compared to the angle made by the laterally-facing (trailing) surfaces of the individual threads 791. The laterally-facing surfaces of the individual threads 791 of compression screw 750 are tilted to be further from being perpendicular to the central axis of the compression screw. In this way, the individual threads 791 “lean” medially. In a complimentary manner, the individual threads 794 of the threaded end portion 779 of the locking element 770 “lean” laterally so that their laterally-facing surfaces are closer to perpendicular to the central axis of compression screw 750, which is used for reference in this context due to the engagement of the threaded end portion 779 of the locking element 770 with the individual threads 791 of compression screw 750. The medially-facing surfaces of the individual threads 794 of the threaded end portion 779 are tilted to be further from being perpendicular to the central axis of the compression screw 750. This asymmetry is shown in FIG. 16A wherein a rectangular box is overlaid on the image to show the relative angles of the faces of the threads on both compression screw 750 and threads 779a on locking element 770. Based on this configuration, the complimentary surfaces of the individual threads 791 and 794 that engage when the compression screw 750 is rotated clockwise to advance it medially with respect to the locking element 770 are closer to vertical, which creates a more secure transfer of force to avoid stripping of any of the screw threads during use of the assembly 7. While this asymmetric threading is shown in assembly 7, it is also contemplated to use assembly 7 with symmetrical threading on both the screw threads on the compression screw 750 and the threaded end portion 779 of the locking element 770.

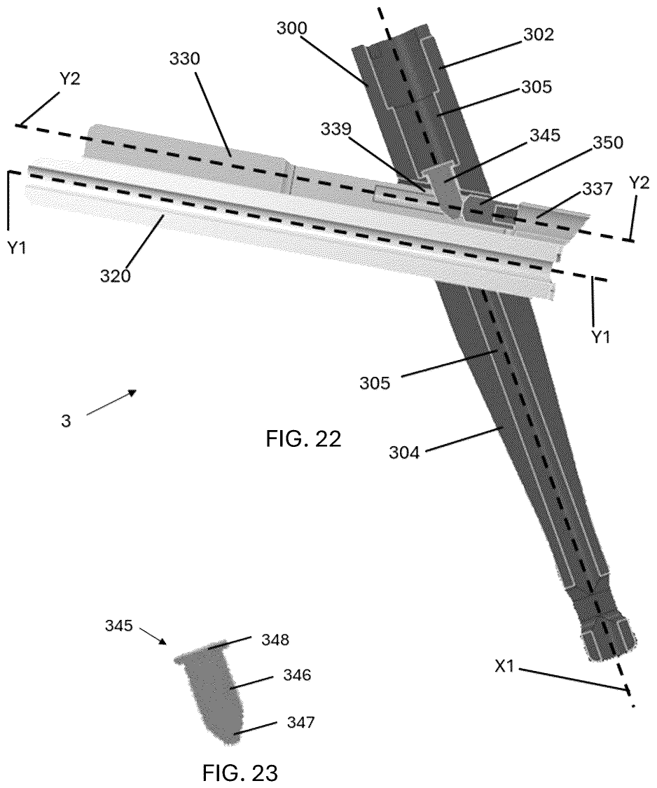

Now referring to FIGS. 22 and 23, another aspect of the present disclosure relates to a fixation assembly 3 that includes intramedullary nail 300, lag screw 320, bolt 230, set screw 345, and compression screw 350. Unless otherwise described, it should be understood that the 300-series reference numerals correspond with the previous series'references numerals to indicate components having the same or similar features as described above. Intramedullary nail 300 has a proximal portion 302 and a distal portion 304 that is tapered. Like with first fixation assembly 1, proximal portion 302 of nail 300 defines a lag screw bore 311 and a bolt bore 312 that extend generally transversely therethrough along a lag screw bore axis Y1, bolt bore axis Y2, respectively. Similar to lag screw bore 111 and bolt 112 described above, lag screw bore 311 and bolt bore 312 intersect to form a double-bore slot 314. Intermedullary nail 300 further defines a cannulated channel 305 that extends through the length of the nail. Lag screw 320, bolt 330 and compression screw 350 have the same or similar features as lag screw 120, bolt bore 130 and compression screw 150, respectively, such that bolt 330 defines a side slot 339 and a cylindrical cavity 337. However, set screw 345 defines a cylindrical shaft 346 having a tapered tip 347 and a head portion 348 that extends beyond the diameter of the shaft. In some instances, set screw 345 is not cannulated.

Fixation assembly 3 is assembled in the same or similar manner as described above for the first fixation assembly 1. Additionally, similar to the first fixation assembly 1, lag screw 320 and bolt 330 of the fixation assembly 3 are static with respect to movement toward a femoral head but dynamic with respect to movement away from the femoral head along lag screw bore axis Y1 and bolt bore axis Y2, respectively.

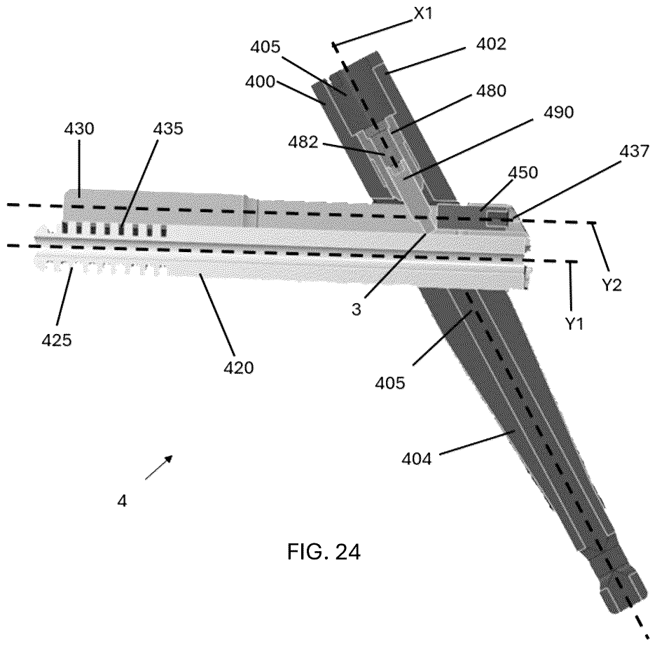

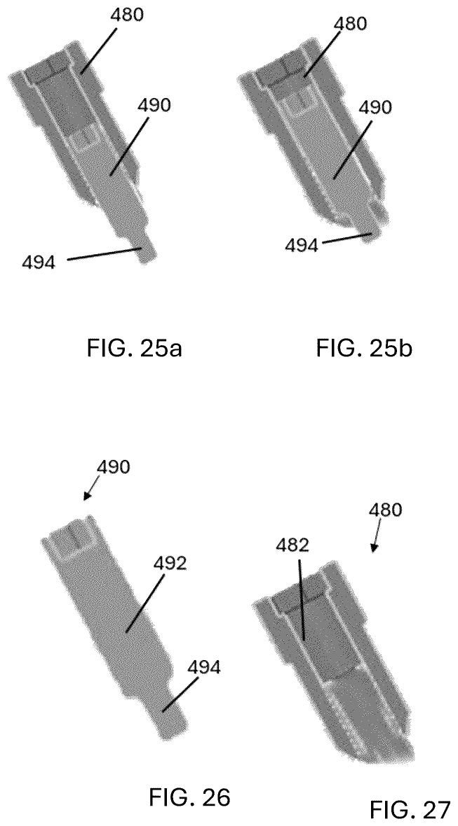

Now referring to FIGS. 24-27, another aspect of the present disclosure relates to a fixation assembly 4 that includes intramedullary nail 400, lag screw 420, bolt 430, compression screw 450, and screw element 480 and locking element 490. Unless otherwise described, it should be understood that the 400-series reference numerals correspond with the previous series'references numerals to indicate components having the same or similar features as described above. For example, nail 400 has a proximal portion 402 that defines a lag screw bore 411 and a bolt bore 412 and a distal portion 404 that is tapered. Similar to lag screw bore 111 and bolt 112 described above, lag screw bore 411 and bolt bore 412 intersect to form a double-bore slot 414. Nail 400 further defines a cannulated channel 405 that extends through the length of the nail. Lag screw 420, bolt 430 and compression screw 450 have the same or similar features as lag screw 120, bolt 130, and compression screw 150, respectively, such that bolt 420 defines a side slot 439 and a cylindrical cavity 437. Locking element 490 defines a cylindrical shaft 492 having a tip 494 extending therefrom. Screw element 480 is cannulated such that it defines a hole 482 extending through the length of the screw element 480 and defining an inner diameter that is larger than the outer diameter of the cylindrical shaft 492 of the locking element 490 such that the locking element can be disposed within screw element 480, as shown in FIGS. 25a and 25b.

Screw element 480 has an internal thread and an external thread, the internal thread is disposed within hole 482. Screw element 480 may be introduced into the body pre-installed within nail 600. The external thread on screw element 480 may be screwed down into bolt 430 to build up pressure with compression screw 450. This will push bolt 430 and lag screw 420 evenly to the right (laterally) of nail 600 as shown in FIG. 24. Optionally, locking element 490 may be screwed down within screw element 480 to point 3 such that the locking element engages lag screw 420 to create a static lock. In this manner, rotation protection is provided as locking element presses against lag screw 420. In some instances, locking element 490 is not pre-installed within screw element 480 and is not cannulated.

Fixation assembly 4 is assembled in the same or similar manner as the first fixation assembly 1 with some exceptions relating to set screw element 480 and locking element 490. For example, set screw element 480 is disposed within cannulated channel 405 and locking element 490 extends from set screw element 480 to cylindrical cavity 437 of bolt 430 to abut compression screw 450 disposed within cylindrical cavity 437 of bolt 430. It is contemplated that side slot 439 may extend entirely through the thickness of bolt 430 to allow for locking element 490 to extend through the bolt to engage lag screw 420 and thereby lock the lag screw in place. Similar to the first fixation assembly 1, lag screw 420 and bolt 430 of fixation assembly 4 are static with respect to movement toward and/or away from a femoral head along lag screw bore axis Y1 and bolt bore axis Y2, respectively.

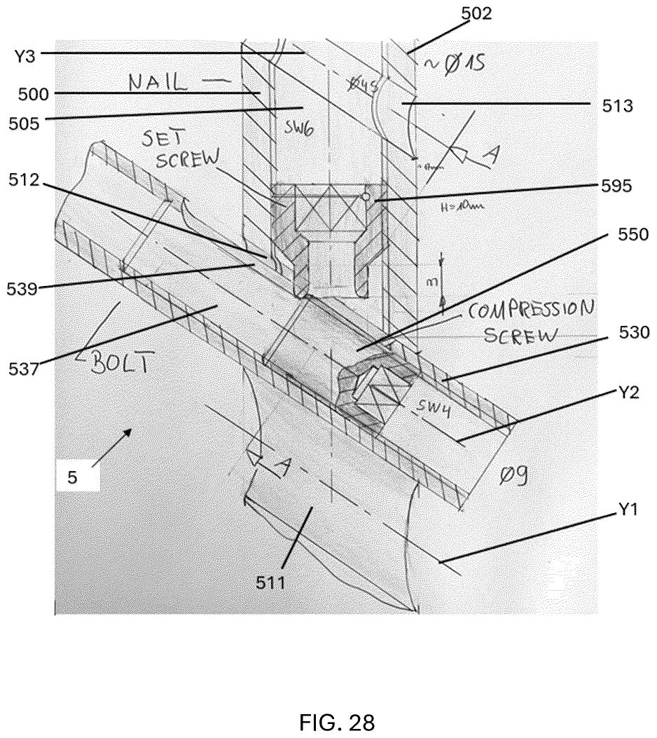

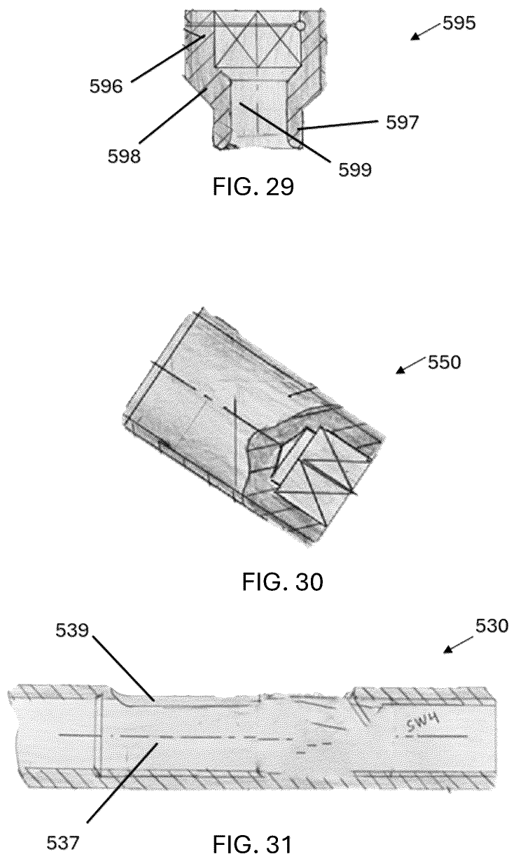

Now referring to FIGS. 28-31, another aspect of the present disclosure relates to a fixation assembly 5 that includes intramedullary nail 500, lag screw (not shown), bolt 530, set screw 595, and compression screw 550. Unless otherwise described, it should be understood that the 500-series reference numerals correspond with the previous series'references numerals to indicate components having the same or similar features as described above. Intramedullary nail 500 has a proximal portion 502 and a distal portion (not shown). Like with first fixation assembly 1, proximal portion 502 of nail 500 defines a lag screw bore 511, a bolt bore 512 and a pin bore 513 that extend generally transversely therethrough along a lag screw bore axis Y1, bolt bore axis Y2 and pin bore axis Y3, respectively. Similar to lag screw bore 111, bolt bore 112 and pin bore 113 described above, lag screw bore 511 and bolt bore 512 intersect to form a double-bore slot. Intermedullary nail 500 further defines a cannulated channel 505 that extends through the length of the nail. Lag screw (not shown), bolt 530 and compression screw 550 have the same or similar features as lag screw 120, bolt 130 and compression screw 150, respectively, such that bolt 530 defines a side slot 539 and a cylindrical cavity 537. However, set screw 595 defines a head portion 396 and a tip portion 547 that are connected by a tapered portion 598. Head portion 596 has a larger diameter than tip portion 597. Set screw 595 further defines a center channel 599 extending through the length of the set screw.

Fixation assembly 5 is assembled in the same or similar manner as described above for the first fixation assembly 1. Additionally, similar to the first fixation assembly, lag screw 520 and bolt 530 of the fixation assembly 5 are static with respect to movement toward a femoral head but dynamic with respect to movement away from the femoral head along lag screw bore axis Y1 and bolt bore axis Y2, respectively.

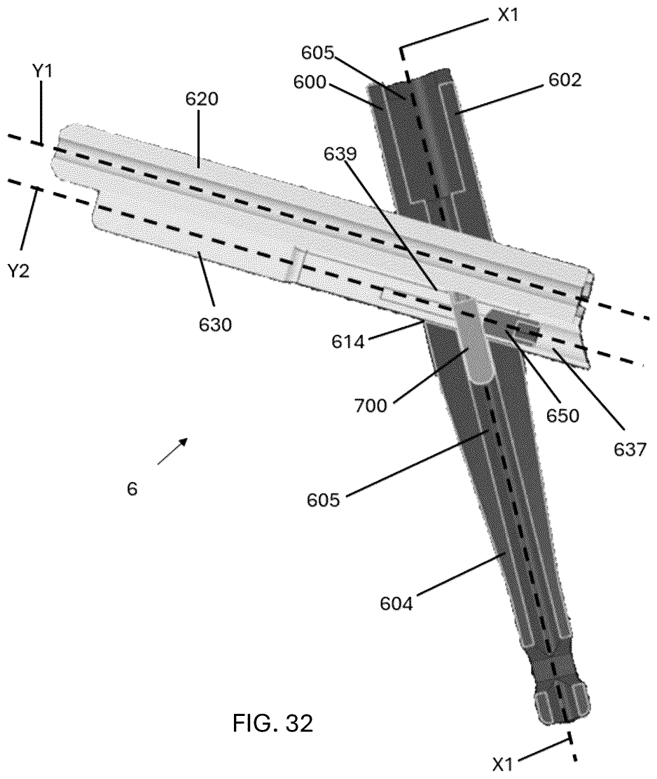

Now referring to FIG. 32, another aspect of the present disclosure relates to a fixation assembly 6 that includes intramedullary nail 600, lag screw 620, bolt 630, set screw 700, and compression screw 650. Unless otherwise described, it should be understood that the 600-series reference numerals correspond with the series'references numerals to indicate components having the same or similar features as described above. Intramedullary nail 600 has a proximal portion 602 and a distal portion 604 that is tapered. Like with the first fixation assembly 1, proximal portion 602 of nail 600 defines a lag screw bore 611 and a bolt bore 612 that extend generally transversely therethrough along a lag screw bore axis Y1, bolt bore axis Y2, respectively. Similar to lag screw bore 111 and bolt 112 described above, lag screw bore 611 and bolt bore 612 intersect to form a double-bore slot 614. Intermedullary nail 600 further defines a cannulated channel 605 that extends through the length of the nail. Lag screw 620, bolt 630 and compression screw 650 have the same or similar features as lag screw 120, bolt bore 130 and compression screw 150, respectively, such that bolt 630 defines a side slot 639 and a cylindrical cavity 637. Side slot 639 extends entirely through the thickness of bolt 630 such that components may be inserted therethrough, e.g., set screw or beveled bolt. Another distinction is that the fixation assembly 6 includes a beveled bolt 700 configured to be disposed in cannulated channel 305 such that the beveled bolt extends through side slot 639 and beneath the double-bore slot 614. In some instances, beveled bolt 649 is not cannulated.

Fixation assembly 6 may be assembled in various ways that include inserting beveled bolt 700 into nail 600 after inserting bolt 630 therethrough. For example, bolt 630 is first pushed through the nail 600. Then beveled bolt 700 is pushed from above through proximal portion 602 of nail 600 and through side slot 639 of bolt 630. Then lag screw 620 is inserted through nail 600 above the bolt 630, as shown in FIG. 32. This can be done so that beveled bolt 700 is in place to engage with a channel in a side surface of lag screw 620 to prevent rotation of lag screw 620 after insertion. Compression screw 650 may then exert tension via a thread, as it can support itself against beveled bolt 700. In this manner, lag screw 620 and bolt 630 of the fixation assembly 6 may provide static locking with respect to movement toward a femoral head but dynamic locking with respect to movement away from the femoral head along lag screw bore axis Y1 and bolt bore axis Y2, respectively.

The components of the fracture fixation assemblies described herein may be included in kits for fixing bone fracture treatment. For example, a kit may include one or more of the intermedullary nails, lag screws, bolts, set screws or swivel screws, locking elements, compression screws, and/or calcar pins disclosed above. The kit may also include tools and instruments designed to implant and assemble the fixation assemblies disclosed herein such as a screwdriver, a reamer instrument, and/or targeting and inserting instruments. Additionally, the components of the fracture fixation assemblies described herein are generally made from biocompatible metals and/or metal alloys such as titanium, stainless steel, and the like.

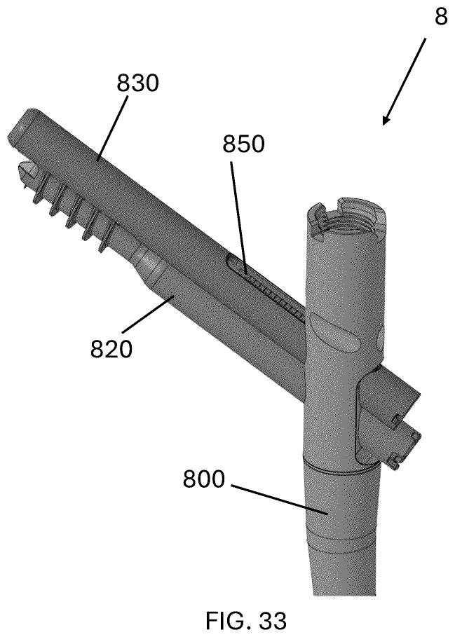

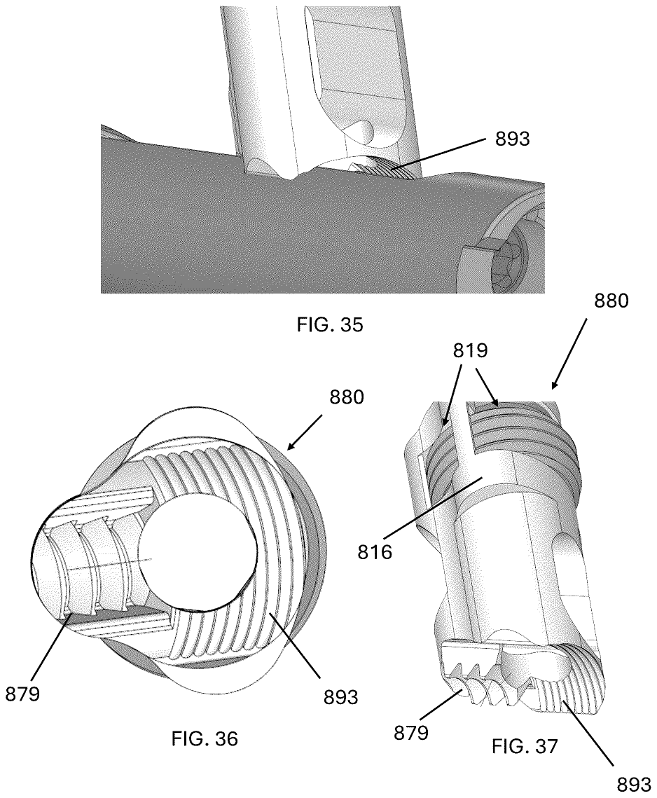

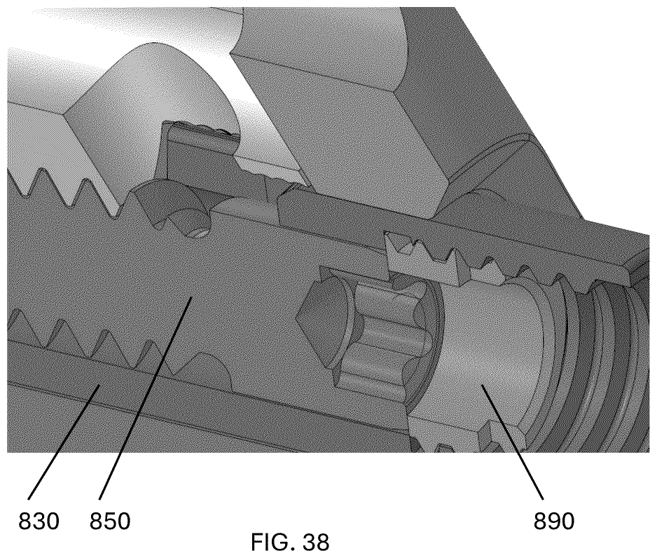

Another aspect of the present disclosure relates to a fixation assembly 8 shown in FIGS. 33-38. Assembly 8 includes intramedullary nail 800, lag screw 820, bolt 830, compression screw 850, swivel screw 860, and locking element 870. Unless otherwise described, it should be understood that the 800-series reference numerals correspond with the previous series'references numerals to indicate components having the same or similar features as described above. Bolt 830 defines a cavity 837 as described above. Compression screw 850 is disposed within cavity 837 so that its threads do not directly engage with the bolt 830. Side slot 839 of bolt 830 permits communication of compression screw 850 with locking element 870.

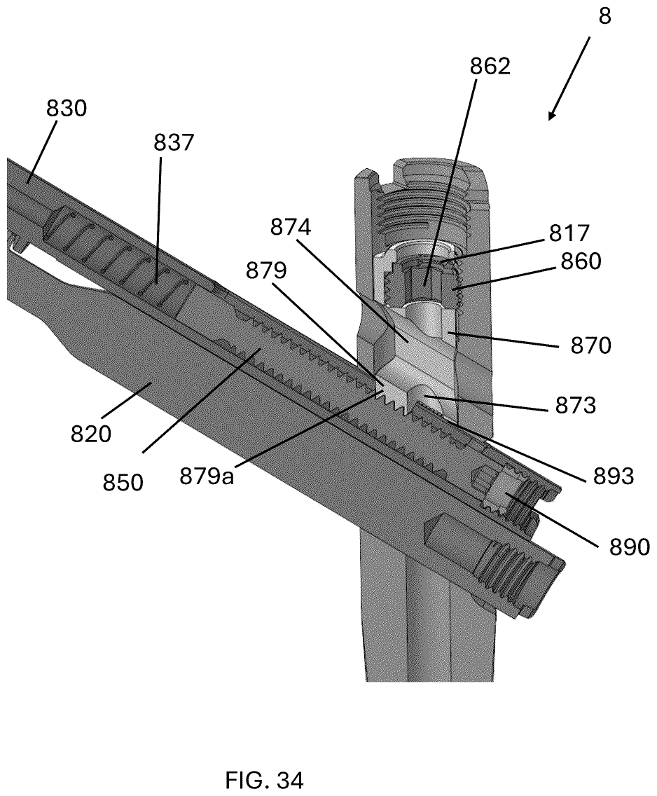

Assembly 8 is similar in nature to assembly 7, in that nail 800 is the same as nail 700, and compression screw 850 is threaded and cooperates with an end screw 890 in the same manner. Set screw 880 is configured differently, as it includes a swivel screw 860 disposed within an upper or proximal portion 816 of locking element 870, as shown in FIGS. 34 and 37. Swivel screw 860 has a substantially cylindrical body provided with an external threading disposed about the body. Swivel screw 860 has a cannulated passage 862 as part of a cannulation completely through set screw 880, and its threaded portion 861 is configured to engage internal threads of nail 800 through one or more lateral windows in the proximal end 816 of locking element 870. Swivel screw 860 sits between upper and lower end walls of portion 861, and within a side wall of portion 861, with the walls defining the windows 819 through which threads of swivel screw 860 extend to contact the nail 800. These upper and lower end walls and the side wall define a cavity in portion 861 in which swivel screw 860 is disposed. Cannulated passage 862 defines hexagonally shaped inner surface arranged to be engaged by insertion and rotation tools. Swivel screw 860 and locking member 870 are rotatably connected to each other to shuttle set screw 880 within nail 800 in the same manner as described above so that swivel screw 860 rotates with respect to nail 800 while locking member 870 does not. Swivel screw 860 may be rotated independent of the locking element 870 allowing the set screw 880 to advance through the nail 800 as the swivel screw is threaded into and advanced along the cannulated channel of the nail 800. Ultimately, swivel screw 860 is sized to be at least partially received within the cavity of locking element 870 in a manner that allows the threading of the swivel screw 860 to protrude from the cavity and to engage the internal threading of nail 800 to threadably mate swivel screw 880 to the nail 800.

In addition, swivel screw 860 includes an elastic member 817 that is transitionable between an expanded condition (e.g., uncompressed) and a compressed condition. Elastic member 817 may be a cantilever arm or flange connected to the remainder of swivel screw 860 at one side. The flange may be formed of any material that exhibits elasticity such as a metal, a metal alloy or a rubber or plastic. In the compressed condition, swivel screw 860 has a length in the axial direction that is equal to or less than the distance between the upper and lower walls of locking element 870. Thus, when elastic member 817 is in the compressed condition, swivel screw 860 can be inserted into the cavity. On the other hand, when elastic member 817 is expanded, the axial length of swivel screw 860 is greater than the distance between the upper and lower walls of portion 861 of locking element 870. As a result, when swivel screw 860 is disposed within the cavity and elastic member 817 is expanded into engagement with the upper and lower walls of locking element 870, the swivel screw is securely coupled to the locking element 870, and lateral movement of the swivel screw relative to the locking element is prevented. The engagement also prevents the swivel screw from unintentionally rotating within the locking element and requires increased torque to intentionally rotate the swivel screw.

Locking element 870 defines a cylindrical channel 873 and an elongated slot 874 as described above. Locking element 870 includes a grooved or threaded end portion 879 at its end that faces compression screw 850. The threads are located only on a leading side of the distal end of locking element 870. Threaded end portion 879 defines a thread pattern 879a configured to engage and interface with the threads of compression screw 850, as shown in FIGS. 34, 36, and 37. The configuration of threaded end portion 879 is similar to portion 779 as described above, though it is only located on the medial side of locking element 870, i.e. on the side of locking element 870 medial of cylindrical channel 873. That is, rather than being divided into two segments, threaded end portion 879 is an isolated threaded segment and makes up just one single medial segment. The configuration of thread pattern 879a is the same as described above in connection with thread pattern 779a, and locking element 870 otherwise cooperates with compression screw 850 in the same manner as compression screw 750 does. Threads of the thread pattern 879a and threads of compression screw 850 can be symmetrical threads, or can be asymmetrical as described above.

Further, locking element 870 includes a concave ridged fixation surface 893 defined by a series of ridges extending in an anterior-posterior direction, i.e. transverse to the longitudinal axis of bolt 830 and compression screw 850. Fixation surface 893 is disposed on a trailing side of the distal end of locking element 870. The threads at threaded end portion 879 occupy a portion of the distal end of locking element 870 that is distinct from a portion of the distal end defining fixation surface 893. In some cases, these distinct portions are separated by cylindrical channel 873. In other words, the ridges extend in generally the same direction as the threads of threaded end portion 879. Ridged surface 893 is configured to press against the adjacent surfaces of bolt 830 with which it comes into contact so that locking element 870 can more securely grip both compression screw 850 and bolt 830 to facilitate static locking of assembly 8. The ridges on ridged surface 893 are relatively sharp, either terminating at a linear ridge or a ridge with a small flat end width of 0.1 mm, to ensure a more complete contact with the relatively smoother surfaces of bolt 830 with which they are exposed. When the ridges interact with the relatively smoother bolt surface, they bite into the bolt surface so that both the ridges and the bolt surface yield to some degree, which enhances frictional locking between locking element 870 and bolt 830. This occurs whether or not locking element 870 and bolt 830 are made of the same material, such as a titanium alloy, or of different materials. Ridged surface 893 does not protrude distally on locking element 870 as far as threaded end portion 879, thus allowing both ridged surface 893 and threaded end portion 879 to mate with bolt 830 and compression screw 850, respectively, simultaneously for more complete locking of assembly 8. Due to the high friction in this statically locked configuration, the movement of the bolt 830 together with the lag screw 820 and head is not possible.

In one embodiment, the angle of the thread faces on the locking element 870 and the compression screw 850 is substantially equal to the angle between the bolt 830 and the nail 800. This allows for the threads on locking element 870 to engage those on compression screw 850 without interference such that a full mating between the threads is facilitated. As locking element 870 is engaged with compression screw, the initial alignment may even force the compression element 850 in a slightly lateral direction.

Fixation assembly 8 may be assembled in a substantially similar way as assembly 7, described above. As the locking element 870 is fixed within the nail 800 in a medial-lateral direction and the compression screw 850 is retained within the bolt 830 by the end screw 890, rotating the compression screw will shift the bolt and the lag screw laterally with respect to the nail. The threads on threaded end portion 879 are the same as those on threaded end portion 779, just in a different overall location. Thus, set screw 880 allows static locking as described above. That static locking is enhanced by the ridged surface 893.

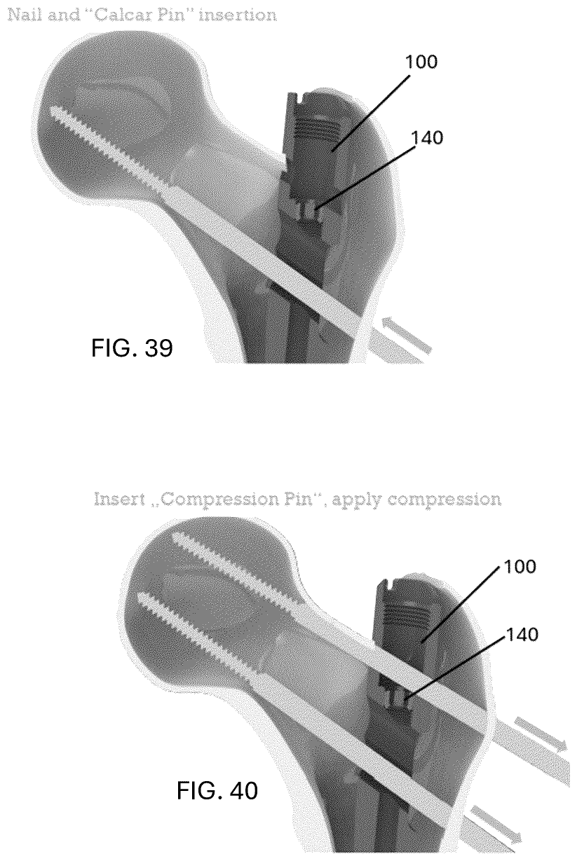

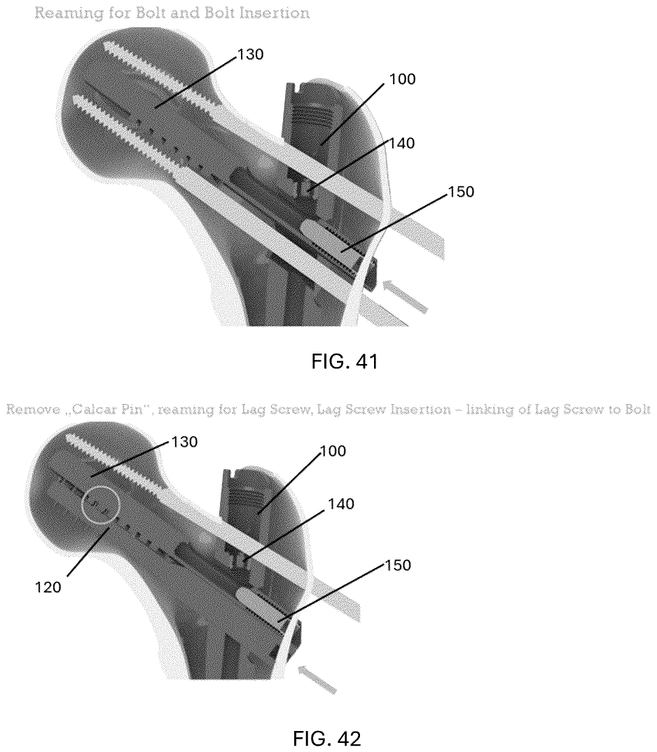

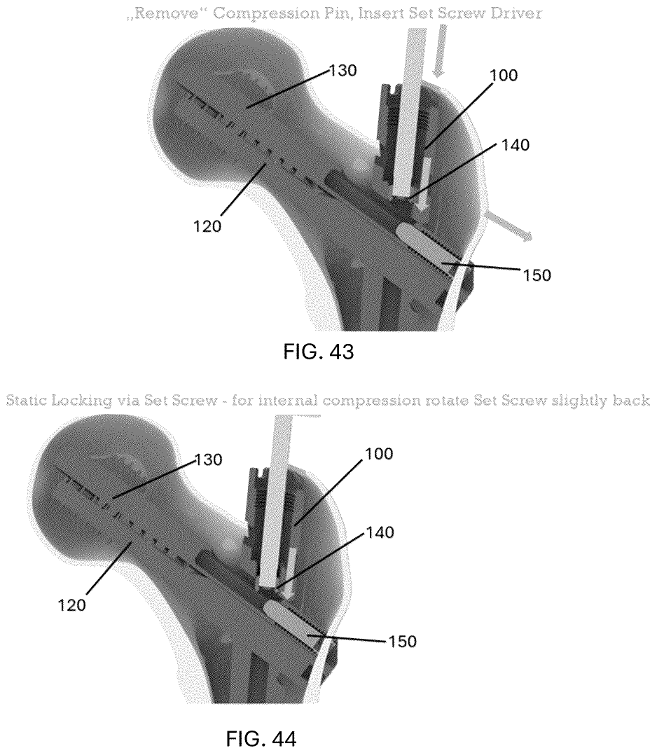

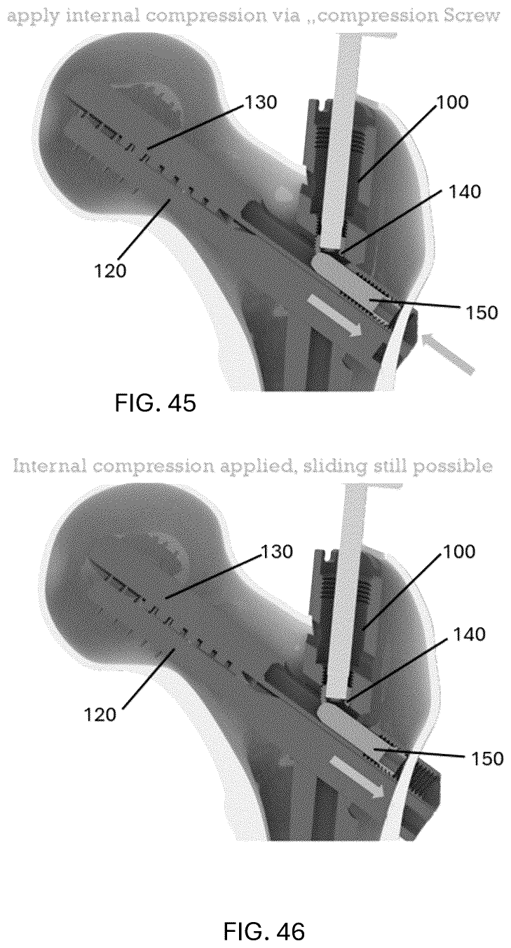

Now referring to FIGS. 39-46, other aspects of the present disclosure relate to methods for assembling and implanting a fixation assembly into a long bone. For example, after a nail (e.g., intramedullary nail 100, 200, 300, 400, 500) has been inserted into an intramedullary canal of a femur, a fixation assembly (e.g., fixation assemblies 1, 2, 3, 4) may be assembled and implanted in the femur by inserting a first calcar pin and a second calcar pin into a lag screw bore (e.g., lag screw bore 111, 211, 311, 411) and a pin bore (e.g., pin bore 113, 213, 313, 413, 513), respectively, such that the first and second calcar pins extend into the head of the femur. Either or both calcar pins can apply compression on the bone fracture and can set the specific location of the femoral head so that the bolt and lag screw can be implanted to the proper depth and location. A set screw (e.g., set screw 140, 345, 595) is threaded into a cannulated channel (e.g., cannulated channel 105, 205, 305, 405, 505) of the nail at least prior to the insertion of the second calcar pin and positioned such that the set screw is located between the first and the second calcar pins once they have been inserted. Next, a bolt (e.g., bolt 130, 230, 330, 430, 530) is inserted through a bolt bore (e.g., bolt bore 112, 212, 312, 412, 512) and into the head of the femur after reaming a bore within the bone, the bolt having a compression screw (e.g., compression screw 150, 250, 350, 450, 550) partially threaded into a cavity (e.g., cylindrical cavity 137, 237, 337, 437, 537) defined by the bolt. The bolt is positioned such that a side slot (e.g., side slot 139, 239, 339, 439, 539) is aligned with the cannulated channel of the nail so that the cylindrical cavity of the bolt is in communication with the cannulated channel via the side slot. Then, after the first calcar pin is removed, a lag screw (e.g., lag screw 120, 220, 320, 420) is inserted into the lag screw bore alongside the bolt such that the threads (e.g., threads 125, 225) are threaded into a threaded path (e.g., threaded path 135, 235) defined by the bolt and into the femoral head. In other words, part of the insertion of the lag screw into the femoral head includes threading the lag screw into the bolt and the anatomy of the femoral head to secure both the lag screw and the bolt thereto. The non-circular construct of the bolt and the lag screw secure and prevent rotation of the femoral head against the remaining portion of the femur during healing. This step is followed by removing the second calcar pin and tightening down or rotating the set screw further into the cannulated channel such that the set screw extends from the cannulated channel and into the cylindrical cavity via the side slot. Then, the compression screw is rotated and advanced further into the cylindrical cavity until the compression screw presses against an end or an edge of the set screw, the set screw blocking further advancement of the compression screw toward the femoral head. Finally, internal compression is applied to the fracture fixation assembly by tightening or rotating the compression screw further into the cylindrical cavity of the bolt. Once the compression screw contacts the set screw, the compression screw cannot advance further into the femur due to the set screw blocking such advancement. Instead, further tightening the compression screw shifts or pulls the bolt and lag screw laterally toward the compression screw and the compression screw advances further into the cylindrical cavity. In some instances, the lag screw may be inserted into the lag screw bore and into the femoral head before removing the first calcar pin by advancing the lag screw over the calcar pin. In such instances, the lag screw has a hollow channel extend through the length of the lag screw and configured to receive a calcar pin. In other instances, only one calcar pin may be used, or the calcar pins may be inserted into different bores defined by the nail.

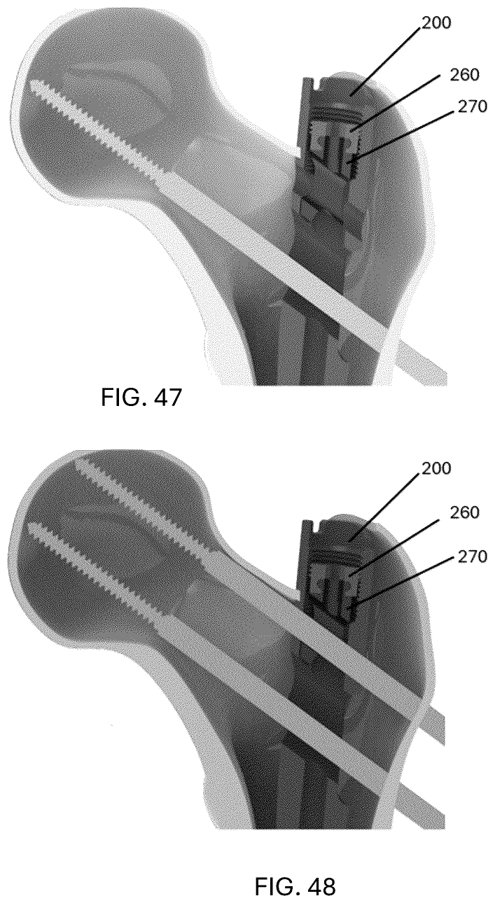

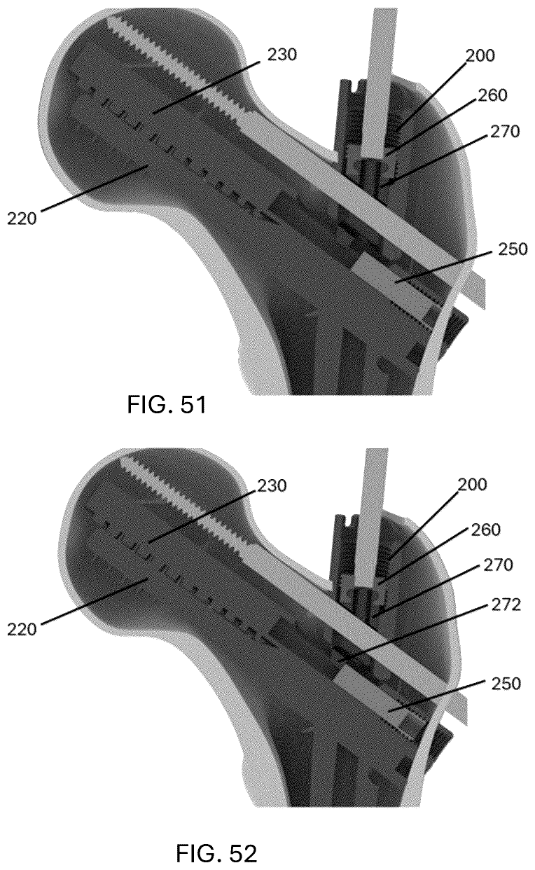

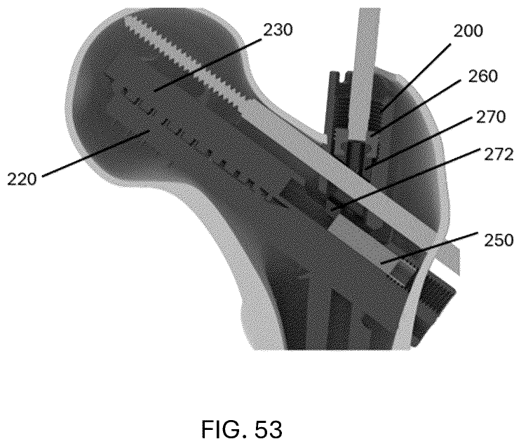

Now referring to FIGS. 47-53, some other aspects of the present disclosure relate to a method for assembling and implanting a fixation assembly into a long bone. For example, a fixation assembly (e.g., fixation assemblies 1, 2, 3, 4, 7, 8) may be assembled and implanted in the femur by inserting a nail (e.g., intramedullary nail 100, 200, 300, 400, 500, 700, 800) into an intramedullary canal of a femur. Either before or after the insertion of the nail, a set screw (e.g., swivel screw 260, 760, 860) rotatably connected to a locking element (e.g., locking element 270, 770, 870) extending distally therefrom is threaded into a cannulated channel (e.g., cannulated channel 105, 205, 305, 405, 505, 705, 805) of the nail. The locking element is positioned within the cannulated channel such that a portion of an elongated slot (e.g., elongated slot 274, 774, 874) of the locking element is aligned with a pin bore (e.g., pin bore 113, 213, 513, 713, 813) defined by the nail, as shown in FIG. 47. Then, a first calcar pin (or some type of compression pin) is inserted into a lag screw bore (e.g., lag screw bore 111, 211, 511, 711, 811) and a second calcar pin (or some type of compression pin) is inserted through a pin bore (e.g., pin bore 113, 213, 513, 713, 813) and thus through the elongated slot defined by the locking element, as shown in FIG. 48. As described above, the elongated slot defines an opening that is larger than the outer diameter of the second calcar pin and the inner diameter of the pin bore. Optionally, the first calcar pin may be inserted before the set screw and the locking element (i.e., the set screw 280, 780, 880) are positioned within the nail. Next, a bolt (e.g., bolt 130, 230, 330, 430, 530, 730, 830) is inserted through a bolt bore (e.g., bolt bore 112, 212, 512, 712, 812) and into the head of the femur, the bolt having a compression screw (e.g., compression screw 150, 250, 350, 450, 550, 750, 850) partially threaded into and/or otherwise located with a cavity (e.g., cylindrical cavity 137, 237, 337, 437, 537, 737, 837) defined by the bolt. The bolt is positioned such that a side slot (e.g., side slot 139, 239, 339, 439, 539, 739, 839) is aligned with the cannulated channel of the nail so that the cylindrical cavity of the bolt is in communication with the cannulated channel via the side slot such that the locking element may be received therein. In most instances, this requires that the bolt be positioned such that its side slot is facing the locking element disposed within the cannulated channel, as shown in FIG. 49. Then, after the removing the first calcar pin, a lag screw (e.g., lag screw 120, 220, 320, 420, 720, 820) is inserted into the first bore alongside the bolt such that the threads (e.g., threads 125, 225, 725, 825) are threaded into a threaded path (e.g., threaded path 135, 235, 735, 835) defined by the bolt and into the femoral head. In other words, part of the insertion of the lag screw into the femoral head includes threading the lag screw into the bolt and the anatomy of the femoral head to secure both the lag screw and the bolt thereto.

Once the bolt and the lag screw have assembled and inserted within the nail, the locking element and compression screw are positioned with respect to each other. For example, the set screw is rotated to thereby advance the locking element further into the cylindrical cavity via the side slot. In some instances, the locking element may include a threaded end portion (e.g., threaded end portion 779 or 879) that is designed to engage the threads on the compression screw. In such instances, the locking element is advanced until the threaded end portion interlock with the threads of the compression screw, as shown in FIGS. 16 and 34. In other instances, the compression screw may be rotated and tightened further into the cylindrical cavity until the compression screw presses against a projection (e.g., projection 272) extending from the locking element, as shown in FIG. 52. Next, internal compression is applied to fracture fixation assembly by tightening or rotating the compression screw to shift the bolt and lag screw laterally thereby applying compression on a fracture in the femoral neck. Finally, the second calcar pin is removed after the locking element and the compression screw are properly placed. Because the locking element is capable of being advanced into the cavity of bolt without needing to remove the second calcar pin, this allows for the fixation assembly to be completely assembled and arranged to apply internal compression to a bone fracture before the support of the calcar pin is removed. In this manner, better rotation stability and control of the femoral head is provided during surgery.

The set screw can be tightened against the compression screw to facilitate this compressive movement of compression screw against the set screw. This gives the surgeon the ability to dictate the compressive force applied when the system is implanted. Similar to the other fixation assemblies, once the compression screw contacts the locking element, the compression screw cannot advance further toward the femoral head due to the locking element blocking such advancement. Instead, further tightening of the compression screw shifts or pulls the bolt and lag screw laterally toward the compression screw and thereby applies compression to the fractured bone. In some instances, the lag screw may be inserted into the first bore and into the femoral head before removing the first calcar pin by advancing the lag screw over the calcar pin. In such instances, the lag screw has a hollow channel extending through the length of the lag screw and configured to receive a calcar pin. In other instances, only one calcar pin may be used, or the calcar pins may be inserted into different bores than those described above.

If static locking is desired, the set screw can be tightened securely against the bolt and the compression screw to inhibit all movement of the bolt and the lag screw with respect to the nail. In the case of set screw 860, concave ridged surface 893 is configured to engage with external surfaces of the bolt 830 when static locking is pursued by tightening the set screw 860 securely against the bolt 830 and the compression screw 850 to ensure contact is made with the compression screw 850 in addition to the surfaces of the bolt 830. Additionally, the width or diameter of the cavity 837 of the bolt 830 is larger than the major diameter of the threads of the compression screw 850 so that the threads of the compression screw do not directly engage with the bolt 730, but also so that the compression screw 850 can bow slightly up to and until the threads of the compression screw 850 contact the bottom surface of cavity 837. This enhances the overall fixation of assembly 8.

The systems of the present application facilitate reduction of femoral neck fractures and compression to maintain the reduction postoperatively. The systems act to oppose or prevent medialization of the lag screw and bolt well permitting sliding of these elements in the lateral direction. In some cases, the leg screw and bolt can be completely locked in place with respect to the nail to prevent any movement, enabling static locking of the system. This is in addition to providing rotational stability to the femoral head during healing.

It is to be understood that the disclosure set forth herein includes any possible combinations of the particular features set forth above, whether specifically disclosed herein or not. For example, where a particular feature is disclosed in the context of a particular aspect, embodiment, arrangement, or configuration, that feature can also be used to the extent possible, in combination with and/or in the context of other particular aspects, embodiments, arrangements, and configurations of the technology, and in the technology in general.

Furthermore, although the technology here has been described with reference to particular features and figures, it is to be understood that these features are merely illustrative of the principles and applications of the present technology. It is therefore to be understood that numerous modifications, including changes in the sizes of the various features described herein, may be made to the illustrative arrangement and that other arrangements may be devised without departing from the spirit and scope of the present technology. In this regard, the present technology encompasses numerous additional features in addition to those specific features set forth in the claims below. Moreover, the foregoing disclosure should be taken by way of illustration rather than by way of limitation as the present technology is defined by the claims set forth below.

1. A fracture fixation system, comprising:
an intramedullary nail having a lag screw bore extending along a lag screw bore axis and a cannulated channel extending along a cannulated channel axis and in communication with the lag screw bore, the lag screw bore axis and the cannulated channel axis extending transverse to each other;
a threaded lag screw configured for insertion within a first portion of the lag screw bore;
a bolt configured for insertion within a second portion of the lag screw bore;
a compression screw extending within the bolt; and
a set screw configured for insertion into the cannulated channel and into engagement with the compression screw and/or the bolt.

2. The fracture fixation system of claim 1, wherein the set screw is disposed closer to the bolt than to the lag screw.

3. The fracture fixation system of claim 1, wherein the bolt includes an aperture extending along a longitudinal axis of the bolt.

4. The fracture fixation system of claim 3, wherein the aperture is at least partially threaded and configured to receive the compression screw through an end of the bolt.

5. The fracture fixation system of claim 1, wherein the set screw includes a set screw body and a locking element.

6. The fracture fixation system of claim 5, wherein the set screw body is rotatably attached to the locking element.

7. The fracture fixation system of claim 5, wherein at least a portion of the set screw body is disposed within a cavity of the locking element.

8. The fracture fixation system of claim 5, wherein the set screw body and the locking element are cannulated.

9. The fracture fixation system of claim 8, wherein the locking element defines an elongated slot that is obliquely oriented with respect to a longitudinal axis of the locking element, and the intramedullary nail further defines a calcar pin bore disposed between the lag screw bore and a proximal end of the intramedullary nail.

10. The fracture fixation system of claim 9, wherein a width of the elongated slot of the locking element is larger than a width of the calcar pin bore.

11. The fracture fixation system of claim 10, wherein the calcar pin bore is aligned with a first portion of the elongated slot of the locking element when the locking element is in a first position in the cannulated channel and with a second portion of the elongated slot of the locking element when the locking element is in a second position in the cannulated channel.

12. The fracture fixation system of claim 9, wherein the cannulated channel has a threaded region and an unthreaded region, and wherein the calcar pin bore extends through the unthreaded region of the cannulated channel between the threaded region and the lag screw bore.

13. The fracture fixation system of claim 5, wherein the locking element is configured to engage the compression screw.

14. The fracture fixation system of claim 5, wherein a distal end of the locking element includes threads configured to engage threads of the compression screw.

15. The fracture fixation system of claim 14, wherein the threads on the distal end of the locking element are located only on a leading side of the distal end of the locking element.

16. The fracture fixation system of claim 15, wherein a trailing side of the distal end of the locking element includes a concave fixation surface configured to engage an exterior surface of the bolt.

17. The fracture fixation system of claim 15, wherein a trailing side of the distal end of the locking element includes a fixation surface, the fixation surface being configured to engage an exterior surface of the bolt, and wherein the threads on the leading side of the distal end of the locking element occupy a portion of the distal end that is distinct from a portion of the distal end defining the fixation surface.

18. The fracture fixation system of claim 15, wherein a trailing side of the distal end of the locking element includes a fixation surface, the fixation surface being configured to engage an exterior surface of the bolt, and wherein when the locking element is engaged with the compression screw, the threads on the leading side of the distal end of the locking element contact the compression screw and the fixation surface on the trailing side of the distal end of the locking element engages the exterior surface of the bolt and cannot contact the compression screw.

19. The fracture fixation system of claim 1, wherein the cannulated channel extends through an entire length of the intramedullary nail.

20. The fracture fixation system of claim 1, wherein the bolt includes a thread path into which the lag screw is threaded.
